(19) United States
(12) Patent Application Publication
(10) Pub. No.: US 2014/0323590 A1
(43) Pub. Date: Oct. 30, 2014

(54) POWDER TREATMENT AGENT COMPRISING SUGAR ALCOHOL-MODIFIED ORGANOPOLYSILOXANE

(71) Applicant: Dow Corning Toray Co., Ltd., Tokyo (JP)

(72) Inventors: Tomohiro IIMURA, Sodegaura-shi (JP); Seiki TAMURA, Ichihara-shi (JP); Tatsuo SOUDA, Ichihara-shi (JP); Akito HAYASHI, Ichihara-shi (JP); Sayuri SAWAYAMA, Ichihara-shi (JP); Haruhiko FURUKAWA, Chiba-shi (JP)

(21) Appl. No.: 14/263,968

(22) Filed: Apr. 28, 2014

Related U.S. Application Data
(62) Division of application No. 13/643,780, filed on Dec. 17, 2012, now abandoned.

(30) Foreign Application Priority Data
Apr. 30, 2010 (JP) 2010-105888

(51) Int. Cl. A61K 8/892; A61Q 19/00

(52) U.S. Cl. CPC A61K 8/892; A61Q 19/00
USPC 514/772

(57) ABSTRACT

The present invention has an objective to provide a surface treatment agent which is safe with no generation of hydrogen, can be suitably used in a surface treatment of powder(s), exhibits good compatibility with other raw materials of cosmetics, and therefore, can improve a dispersing property and stability of powder(s) contained in a cosmetic, as well as provide a powder which has been subjected to a surface treatment with the aforementioned powder treatment agent, and a cosmetic raw material and a cosmetic which are produced by blending them, exhibit superior storage stability, have a superior outer appearance, and exhibit a superior sensation during use. A sugar alcohol-modified organopolysiloxane having a specified structure is used in the powder treatment.

### RELATED APPLICATIONS

This application is a divisional of U.S. patent application Ser. No. 13/643,780, filed on Oct. 26, 2012, which is the National Stage of International Patent Application No. PCT/JP2011/060800, filed on Apr. 28, 2011, which claims priority to and all the advantages of Japanese Patent Application No. 2010-105888, filed on Apr. 30, 2010, the contents of which are incorporated herein by reference.

### TECHNICAL FIELD

The present invention relates to a cosmetic. In particular, the present invention relates to a powder treatment agent comprising a sugar alcohol-modified organopolysiloxane, a powder surface-treated with the aforementioned powder treatment agent, and a cosmetic raw material and cosmetic comprising the same.

### BACKGROUND ART

Various types of powders represented by white and colored pigments such as titanium oxide, zinc oxide, red iron oxide and the like, as well as those represented by extender pigments such as mica, sericite and the like have been widely used in the field of various cosmetics such as a sunscreen, a nail color, a nail coating, a foundation, a mascara, an eye liner and the like, as well as skin care cosmetics. However, in untreated powders, clumping easily occurs due to caused by charges and polarities of the surface of the powders, trace amounts of impurities, and the like. For this reason, in order to improve a dispersing property and stability of powders in a cosmetic, and in order to improve feeling on touch, water resistance, sebum resistance of a cosmetic containing powders, and the like, use of powders which are subjected to various surface treatments has been carried out.

As the aforementioned surface treatments, a lipophilic treatment using an oil agent, a metal soap or the like, a hydrophilic treatment using a surfactant, a water-soluble polymer or the like, a hydrophobic treatment using a silicone compound, a silica treatment, an alumina treatment and the like have been known. In particular, recently, a surface treatment with a silicone compound having a reactive moiety in a molecule is carried out in many cases. The aforementioned reactive moiety forms a chemical bond with the surface of powders, and for this reason, the surface treatment with the aforementioned silicone compound is effective in view of modification of the surface of powders and at the same time, blocking a surface activity of the powders. In addition, the surface treatment can be reliably carried out, and for this reason, even in the case of blending the surface-treated powders in a cosmetic containing a solvent, the surface treatment agent is not removed from the surface of the powders. In addition, changes in the properties of powders caused by the surface treatment, can be minimized. As examples of the aforementioned surface treatments, mention may be made of, for example, a method in which a surface treatment of powders is carried out with a methylhydrogenpolysiloxane (JP-B-2719303). However, in the aforementioned methods, an unreacted Si—H group remains in powders even after the surface treatment is carried out. Therefore, in the case of blending the surface-treated powders in a cosmetic, there may be a problem such as generation of a hydrogen gas caused by a specific component blended in the cosmetic in some cases.

On the other hand, a method for producing a dispersion of powders using an organopolysiloxane modified with a hydrophilic group which is compatible with the surface of the powders has been proposed. For example, a method in which a polyether-modified silicone is used as a dispersion auxiliary agent of powders has been proposed (JP-A-H10-167946). However, the effects of dispersing the powders are not sufficient. For this reason, there is a problem in that the viscosity of a dispersion of powders obtained by dispersing powders in an oil agent such as a silicone oil generally increases over time, and fluidity is impaired.

In addition, a method in which an organopolysiloxane modified with a polyhydric alcohol such as a (poly)glycerol is used as a hydrophilic group has been proposed (JP-A-2002-38013). However, the dispersing property of the powders in the dispersion is not sufficient, and there is a problem in fluidity of the dispersion obtained as a result.

In addition, an organopolysiloxane modified with a sugar as a hydrophilic group, and an application thereof to a cosmetic have been proposed (JP-A-H05-186596, JP-A-2002-119840 and JP-A-2008-274241). However, an application thereof to a powder treatment has not been disclosed at all therein.

### DISCLOSURE OF INVENTION

### Technical Problems

The present invention has been made in view of the circumstances of the aforementioned prior art. The objective of the present invention is to provide a surface treatment agent which is safe with no generation of hydrogen, can be suitably used in a surface treatment of powders, can exhibit good compatibility with other raw materials of cosmetics, and therefore, can improve dispersing properties and stability of powders contained in a cosmetic, and in addition, can provide good water resistance, good sebum resistance, good glossiness, good feeling on touch, good adhesive properties, and the like to a cosmetic, as well as to provide a powder which has been subjected to a surface treatment with the aforementioned powder treatment agent, and a cosmetic raw material and a cosmetic which can be produced by blending them, can exhibit superior storage stability, can have a superior outer appearance, and can exhibit a superior sensation during use.

### Technical Solution

The objective of the present invention can be achieved by a powder treatment agent comprising a sugar alcohol-modified organopolysiloxane in which a sugar alcohol-containing organic group represented by the following general formula (1-1):

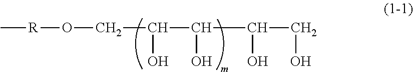

wherein
R represents a divalent organic group; and
m is 1 or 2,
or represented by the following general formula (1-2):

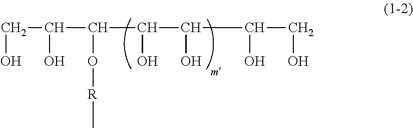

wherein
R is the same as defined above; and
m′ is 0 or 1,
is bonded to a silicon atom.

In the aforementioned general formula (1-1) or (1-2), the divalent organic group, which is R, is preferably a substituted or non-substituted, and linear or branched divalent hydrocarbon group having 3 to 5 carbon atoms.

The aforementioned sugar alcohol-modified organopolysiloxane is preferably represented by the following average compositional formula:

XₓR¹_{y}SiO_{(4-x-y)/2}

wherein
X represents a sugar alcohol-containing organic group represented by the aforementioned general formula (1-1) or (1-2);
R¹ represents a monovalent organic group, with the proviso that the one represented by X is excluded therefrom; and
x and y are the numbers independently satisfying 0<x<1, 0<y<3, and 0<x+y<4.

The monovalent organic group, which is R¹ in the aforementioned average compositional formula, preferably represents a substituted or non-substituted, and linear or branched monovalent hydrocarbon group having 1 to 8 carbon atoms, a polyoxyalkylene group represented by the following formula: —R²O(AO)ₙR³ wherein AO represents an oxyalkylene group having 2 to 4 carbon atoms; R² represents a substituted or non-substituted, and linear or branched divalent hydrocarbon group having 3 to 5 carbon atoms; R³ represents a hydrogen atom, a substituted or non-substituted, and linear or branched monovalent hydrocarbon group having 1 to 8 carbon atoms, or a substituted or non-substituted, and linear or branched acyl group having 2 to 8 carbon atoms; and n=1 to 100, an alkoxy group having 1 to 8 carbon atoms, a hydroxyl group or a hydrogen atom, with the proviso that all R¹s do not represent a hydroxyl group, a hydrogen atom, the aforementioned alkoxy group or the aforementioned polyoxyalkylene group.

The aforementioned sugar alcohol-modified organopolysiloxane is preferably represented by the following general formula (2):

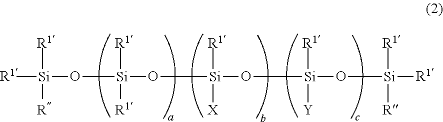

wherein
X represents a sugar alcohol-containing organic group represented by the aforementioned general formula (1-1) or (1-2);
R^{1′} represents a substituted or non-substituted, and linear or branched monovalent hydrocarbon group having 1 to 8 carbon atoms;
Y represents a polyoxyalkylene group represented by the following formula: —R⁴O(AO)ₙR⁵ wherein AO represents an oxyalkylene group having 2 to 4 carbon atoms;
R⁴ represents a substituted or non-substituted, and linear or branched divalent hydrocarbon group having 3 to 5 carbon atoms; R⁵ represents a hydrogen atom, a substituted or non-substituted, and linear or branched monovalent hydrocarbon group having 1 to 8 carbon atoms, or a substituted or non-substituted, and linear or branched acyl group having 2 to 8 carbon atoms; and n=1 to 100;
R″ represents any one of the functional groups represented by the aforementioned R^{1′}, X and Y;
a=0 to 700; b=0 to 100; and c=0 to 50, with the proviso that in the case of b=0, at least one R″ is X.

The aforementioned sugar alcohol-modified organopolysiloxane is, in particular, preferably represented by the following general formula (2′):

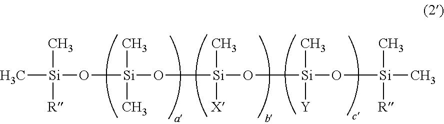

wherein
X′ represents a sugar alcohol-containing organic group represented by the following general formula (1′-1):

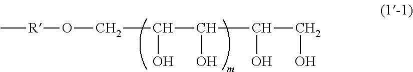

wherein
R′ represents a substituted or non-substituted, and linear or branched alkylene group having 3 to 5 carbon atoms; and m is 1 or 2,
or represented by the following general formula (1′-2):

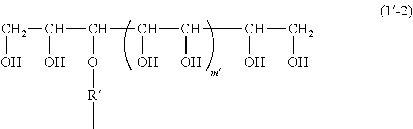

wherein
R^{1′} is the same as defined above; and m′ is 0 or 1;
Y represents a polyoxyalkylene group represented by the following formula: —R⁴O(AO)ₙR⁵ wherein AO represents an oxyalkylene group having 2 to 4 carbon atoms;
R⁴ represents a substituted or non-substituted, and linear or branched divalent hydrocarbon group having 3 to 5 carbon atoms; R⁵ represents a hydrogen atom, a substituted or non-substituted, and linear or branched monovalent hydrocarbon group having 1 to 8 carbon atoms, or a substituted or non-substituted, and linear or branched acyl group having 2 to 8 carbon atoms; and n=1 to 100
R″ represents any one of a methyl group, a group represented by the aforementioned X′ and a group represented by the aforementioned Y;
a′=0 to 250; b′=0 to 50; c′=0 to 25; and a′+b′+c′=0 to 250, with the proviso that in the case of b′=0, at least one R″ is X′.

The aforementioned powder treatment agent is preferably a powder-surface treatment agent.

The present invention also relates to a powder which has been subjected to a surface treatment with the aforementioned powder treatment agent.

In addition, the present invention also relates to a cosmetic raw material comprising the aforementioned powder treatment agent and powder, and further optionally comprising at least one oil agent, which is in the form of a liquid at 5 to 100° C., and is selected from the group consisting of a silicone oil, a non-polar organic compound and a low-polar organic compound.

In addition, the present invention also relates to a cosmetic comprising the aforementioned surface-treated powder or the aforementioned cosmetic raw material.

The cosmetic of the present invention can further contain at least one oil agent, which is in the form of a liquid at 5 to 100° C., and is selected from the group consisting of a silicone oil, a non-polar organic compound and a low-polar organic compound.

The cosmetic of the present invention can further contain water.

The cosmetic of the present invention is preferably in the form of an oil-in-water emulsion or a water-in-oil emulsion.

### Advantageous Effects of Invention

The powder treatment agent of the present invention can be, in particular, preferably used in powders for use in a cosmetic. In the present invention, hydrogen is not generated, and for this reason, the powder treatment agent is safe. In addition, the powder treatment agent of the present invention uses a polymer in which a hydrophilic sugar alcohol is grafted on the hydrophobic main chain, and for this reason, good compatibility with various other components which are hydrophilic or hydrophobic in a cosmetic can be exhibited. Therefore, a dispersing property and stability of powders contained in a cosmetic can be enhanced, and as a result, stability of the cosmetic can also be enhanced. In addition, the powder treatment agent and powder which has been subjected to a surface treatment with the aforementioned powder treatment agent of the present invention can provide good water resistance, good sebum resistance, good glossiness, a good feeling on touch, a good adhesive property and the like to a cosmetic.

Therefore, the cosmetic of the present invention can exhibit superior storage stability, can has a superior outer appearance, and can exhibit a superior sensation during use. In particular, the cosmetic can be superior in view of water resistance, sebum resistance, glossiness, feeling on touch, an adhesive property with respect to hair, skin and the like.

### BEST MODES FOR CARRYING OUT THE INVENTION

The powder treatment agent of the present invention comprises a sugar alcohol-modified organopolysiloxane in which a sugar alcohol-containing organic group represented by the following general formula (1-1):

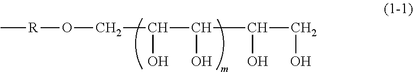

wherein
R represents a divalent organic group; and
m is 1 or 2,
or represented by the following general formula (1-2):

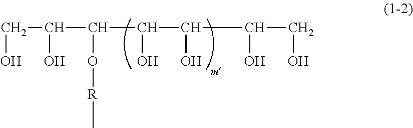

wherein
R is the same as defined above; and
m′ is 0 or 1,
is bonded to a silicon atom.

The sugar alcohol-modified organopolysiloxane according to the present invention is characterized by binding at least one type of the aforementioned organic groups containing a sugar alcohol group represented by the aforementioned general formulae (1-1) or (1-2) to a silicon atom. In addition, the sugar alcohol-modified organopolysiloxane may be an organopolysiloxane having two or more types selected from the aforementioned organic groups containing a sugar alcohol group in one molecule. In the same manner as described above, the sugar alcohol-modified organopolysiloxane may be a mixture of organopolysiloxanes having different types of organic groups containing a sugar alcohol group.

The divalent organic group is not particularly restricted, and as an example thereof, mention may be made of a substituted or non-substituted, and linear or branched divalent hydrocarbon group having 1 to 8 carbon atoms. A substituted or non-substituted, and linear or branched divalent hydrocarbon group having 3 to 5 carbon atoms is preferred. As examples of the substituted or non-substituted, and linear or branched divalent hydrocarbon group having 1 to 8 carbon atoms, mention may be made of, for example, linear or branched alkylene groups having 1 to 8 carbon atoms such as a methylene group, a dimethylene group, a trimethylene group, a tetramethylene group, a pentamethylene group, a hexamethylene group, a heptamethylene group, an octamethylene group and the like; alkenylene groups having 2 to 8 carbon atoms such as a vinylene, an allylene group, a butenylene group, a hexenylene group, an octenylene group and the like; a phenylene group; alkylenearylene groups having 7 or 8 carbon atoms such as a phenylene group, a dimethylenephenylene group and the like; and substituted groups thereof in which hydrogen atoms binding to carbon atoms of the aforementioned groups are at least partially substituted by a halogen atom such as a fluorine atom or the like. The divalent hydrocarbon group is preferably an alkylene group having 1 to 8 carbon atoms, more preferably an alkylene group having 3 to 5 carbon atoms.

As the sugar alcohol-containing organic group, the case in which R is a propylene group and m=1 in the aforementioned general formula (1-1) is, in particular, preferred. In the same manner as described above, as the sugar alcohol-containing organic group, the case in which R is a propylene group and m′=0 in the aforementioned general formula (1-2) is, in particular, preferred. The sugar alcohol-containing organic group in this case is a xylitol residue (hereinafter, merely referred to as “xylitol residue” or “xylitol-modified group”) represented by the following structural formula: —C₃H₆—OCH₂[CH(OH)]₃CH₂OH or the following structural formula: —C₃H₆—OCH{CH(OH)CH₂OH}₂, respectively in the case of the aforementioned general formula (1-1) or (1-2).

The binding site of the sugar alcohol-containing organic group may be any one of the side chain or the terminal of the polysiloxane which is the main chain. A structure in which two or more sugar alcohol-containing organic groups are present in one molecule of the sugar alcohol-modified organopolysiloxane may be used. In addition, the aforementioned two or more sugar alcohol-containing organic groups may be the same or different sugar alcohol-containing organic groups. A structure in which the aforementioned two or more sugar alcohol-containing organic groups bind to only the side chain(s), only the terminal(s), or both the side chain(s) and the terminal(s) of the polysiloxane which is the main chain may be used.

The aforementioned sugar alcohol-modified organopolysiloxane is preferably represented by the following average compositional formula:

XₓR¹_{y}SiO_{(4-x-y)/2}

wherein
X represents a sugar alcohol-containing organic group represented by the aforementioned general formula (1-1) or (1-2);
R¹ represents a monovalent organic group, with the proviso that one represented by X is excluded therefrom; and
x and y are the numbers independently satisfying 0<x<1, 0<y<3, and 0<x+y<4.

The sugar alcohol-modified organopolysiloxane according to the present invention possesses the sugar alcohol-containing organic group represented by the aforementioned general formula (1-1) or (1-2) in one molecule. The aforementioned sugar alcohol-containing organic groups may be identical to each other or a combination of two or more types thereof. In addition, in order to achieve the purpose of the present invention, a mixture of two or more types of the sugar alcohol-modified organopolysiloxanes which are different may be used.

The monovalent organic group is not particularly restricted, and preferably represents a substituted or non-substituted, and linear or branched monovalent hydrocarbon group having 1 to 8 carbon atoms, a polyoxyalkylene group represented by the following formula: —R²O(AO)ₙR³ wherein AO represents an oxyalkylene group having 2 to 4 carbon atoms; R² represents a substituted or non-substituted, and linear or branched divalent hydrocarbon group having 3 to 5 carbon atoms; R³ represents a hydrogen atom, a substituted or non-substituted, and linear or branched monovalent hydrocarbon group having 1 to 8 carbon atoms, or a substituted or non-substituted, and linear or branched acyl group having 2 to 8 carbon atoms; and n=1 to 100, an alkoxy group having 1 to 8 carbon atoms, a hydroxyl group (—OH), or a hydrogen atom, with the proviso that all R¹s do not represent a hydroxyl group, a hydrogen atom, the aforementioned alkoxy group or the aforementioned polyoxyalkylene group.

As examples of monovalent hydrocarbon groups having 1 to 8 carbon atoms, mention may be made of, for example, alkyl groups such as a methyl group, an ethyl group, a propyl group, a butyl group, a pentyl group, a hexyl group, a heptyl group, an octyl group and the like; cycloalkyl groups such as a cyclopentyl group, a cyclohexyl group and the like; alkenyl groups such as a vinyl group, an allyl group, a butenyl group and the like; aryl groups such as a phenyl group, a tolyl group and the like; aralkyl groups such as a benzyl group and the like; substituted groups thereof in which the hydrogen atoms binding to the carbon atoms of the aforementioned groups are at least partially substituted with a halogen atom such as a fluorine atom or the like, or an organic group containing an epoxy group, a glycidyl group, an acyl group, a carboxyl group, an amino group, a methacryl group, a mercapto group or the like, with the proviso that the total number of the carbon atoms ranges from 1 to 8 carbon atoms. The monovalent hydrocarbon group is preferably a group other than an alkenyl group, and a methyl group, an ethyl group or a phenyl group is, in particular, preferred. In addition, as examples of alkoxy groups having 1 to 8 carbon atoms, mention may be made of lower alkoxy groups such as a methoxy group, an ethoxy group, an isopropoxy group, a butoxy group and the like.

As examples of divalent hydrocarbon groups having 3 to 5 carbon atoms, mention may be made of, for example, alkylene groups such as a trimethylene group, a tetramethylene group, a pentamethylene group, and the like; substituted groups thereof in which the hydrogen atoms binding to the carbon atoms of the aforementioned groups are at least partially substituted with a halogen atom such as a fluorine atom or the like, or an organic group containing an epoxy group, a glycidyl group, an acyl group, a carboxyl group, an amino group, a methacryl group, a mercapto group or the like, with the proviso that the total number of the carbon atoms ranges from 3 to 5 carbon atoms.

The aforementioned sugar alcohol-modified organopolysiloxane is preferably represented by the following general formula (2):

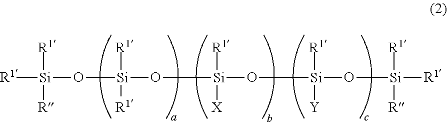

wherein
X represents a sugar alcohol-containing organic group represented by the aforementioned general formula (1-1) or (1-2);
R^{1′} represents a substituted or non-substituted, and linear or branched monovalent hydrocarbon group having 1 to 8 carbon atoms;
Y represents a polyoxyalkylene group represented by the following formula: —R⁴O(AO)ₙR⁵ wherein AO represents an oxyalkylene group having 2 to 4 carbon atoms;
R⁴ represents a substituted or non-substituted, and linear or branched divalent hydrocarbon group having 3 to 5 carbon atoms; R⁵ represents a hydrogen atom, a substituted or non-substituted, and linear or branched monovalent hydrocarbon group having 1 to 8 carbon atoms, or a substituted or non-substituted, and linear or branched acyl group having 2 to 8 carbon atoms; and n=1 to 100
R″ represents any one of the functional groups represented by the aforementioned R^{1′}, X and Y;
a=0 to 700, preferably 0 to 500, and in particular, preferably 0 to 250; b=0 to 100, and preferably 0 to 50; and c=0 to 50, and preferably 0 to 25, with the proviso that in the case of b=0, at least one R″ is X.

The divalent hydrocarbon group having 3 to 5 carbon atoms as R⁴ and the monovalent hydrocarbon group having 1 to 8 carbon atoms as R⁵ are the same as described above.

The chain length of the polysiloxane consisting of diorganosiloxane units is preferably 250 or less in view of the properties as the powder treatment agent. Therefore, in the aforementioned general formula (2), a+b+c is preferably 250 or less.

A modification index of an organopolysiloxane with a sugar alcohol-containing organic group preferably ranges from 1 to 50% by mol, and more preferably ranges from 2.5 to 45% by mol among all functional groups binding to the polysiloxane which is the main chain, in view of providing the properties as the powder treatment agent. In the sugar alcohol-modified organopolysiloxane represented by the aforementioned structural formula (2), the modification index with a sugar alcohol-containing organic group is indicated by the following equation:

Modification index (% by mol)=100×(the number of a sugar alcohol-containing organic groups binding to a silicon atom in one molecule)/{6+2×(a+b+c)}.

For example, in the case of a sugar alcohol-modified organopolysiloxane formed from a trisiloxane possessing one sugar alcohol-containing organic group, one functional group binding to a silicon atom among eight functional groups binding to silicon atoms is modified with a sugar alcohol-containing organic group. For this reason, the modification index with a sugar alcohol-containing organic group is 12.5% by mol.

The aforementioned sugar alcohol-modified organopolysiloxane is, in particular, preferably represented by the following general formula (2′):

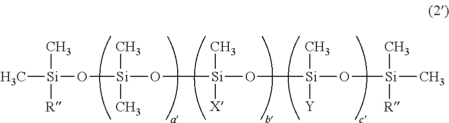

wherein
X′ represents a sugar alcohol-containing organic group represented by the following general formula (1′-1):

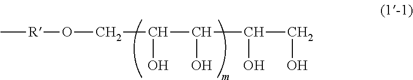

wherein
R′ represents a substituted or non-substituted, and linear or branched alkylene group having 3 to 5 carbon atoms; and m is 1 or 2,
or represented by the following general formula (1′-2):

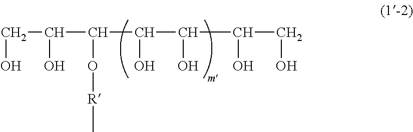

wherein
R′ is the same as defined above; and m′ is 0 or 1,
Y represents a polyoxyalkylene group represented by the following formula: —R⁴O(AO)ₙR⁵, wherein AO represents an oxyalkylene group having 2 to 4 carbon atoms; R⁴ represents a substituted or non-substituted, and linear or branched divalent hydrocarbon group having 3 to 5 carbon atoms; R⁵ represents a hydrogen atom, a substituted or non-substituted, and linear or branched monovalent hydrocarbon group having 1 to 8 carbon atoms, or a substituted or non-substituted, and linear or branched acyl group having 2 to 8 carbon atoms; and n=1 to 100;
R″ represents any one of a methyl group, a group represented by the aforementioned X′, and a group represented by the aforementioned Y;
a′=0 to 250, and preferably 0 to 100; b′=0 to 50, and preferably 0 to 10; and c′=0 to 25, and preferably 0 to 5, in which a′+b′+c′ is the number ranging from 0 to 250 and preferably ranging from 0 to 100, and in the case of b′=0, at least one R″ is X′.

In the aforementioned structural formula (2′), each X′ is independently a sugar alcohol-containing organic group represented by the aforementioned general formula (1′-1) or general formula (1′-2). In the sugar alcohol-modified organopolysiloxane according to the present invention, all X's may be sugar alcohol-containing organic groups represented by the aforementioned general formula (1′-1) or general formula (1′-2), or alternatively, a part of X′ in one molecule may be a sugar alcohol-containing organic group represented by the aforementioned general formula (1′-1), and the remaining X′ may be a sugar alcohol-containing organic group represented by the aforementioned general formula (1′-2).

In addition, the sugar alcohol-modified organopolysiloxane according to the present invention may be one type of the sugar alcohol-modified organopolysiloxane represented by the aforementioned general formula (2′) or a mixture of two or more types thereof.

In particular, in the sugar alcohol-modified organopolysiloxane according to the present invention, represented by the aforementioned general formula (2′), X′ is preferably a sugar alcohol-containing organic group which is a xylitol residue, in view of properties as the powder treatment agent thereof, and in particular, dispersing properties of inorganic powders.

As described above, the xylitol residue is a group represented by the structural formula: —C₃H₆—OCH₂—[CH(OH)]₃CH₂OH or the structural formula: —C₃H₆—OCH{CH(OH)CH₂OH}₂. In the sugar alcohol-modified organopolysiloxane according to the present invention, the aforementioned xylitol residues may be one type or two types. Therefore, in the aforementioned general formula (2′), all X's may consist of only the xylitol residue represented by the structural formula: —C₃H₆—OCH₂[CH(OH)]₃CH₂OH or the structural formula: —C₃H₆—OCH{CH(OH)CH₂OH}₂, or alternatively, X's may consist of two types of xylitol residues represented by the structural formula: —C₃H₆—OCH₂[CH(OH)]₃CH₂OH and represented by the structural formula: —C₃H₆—OCH{CH(OH)CH₂OH}₂. In the latter case, the composition ratio (weight ratio) preferably ranges from 5:5 to 10:0, and in particular, preferably ranges from 8:2 to 10:0. The case of 10:0 means that X′ substantially consists of only a xylitol residue represented by the structural formula:

—C₃H₆—OCH₂[CH(OH)]₃CH₂OH.

In addition, in the case in which the sugar alcohol-modified organopolysiloxane according to the present invention is a mixture of two or more types of sugar alcohol-modified organopolysiloxanes, the aforementioned mixture can comprise at least two types of sugar alcohol-modified organopolysiloxanes selected from the group consisting of a sugar alcohol-modified organopolysiloxane in which X′ in the aforementioned general formula (2′) consists of only a xylitol residue represented by the structural formula: —C₃H₆—OCH₂[CH(OH)]₃CH₂OH, a sugar alcohol-modified organopolysiloxane in which X′ in the aforementioned general formula (2′) consists of only a xylitol residue represented by the structural formula: —C₃H₆—OCH{CH(OH)CH₂OH}₂, and a sugar alcohol-modified organopolysiloxane in which X in the aforementioned general formula (2′) consists of two types of xylitol residues represented by the structural formula: —C₃H₆—OCH₂[CH(OH)]₃CH₂OH and the structural formula: —C₃H₆—OCH{CH(OH)CH₂OH}₂ in a constitutional ratio (weight ratio) preferably ranging from 5:5 to 10:0 and in particular, preferably ranging from 8:2 to 10:0. In addition, the sugar alcohol-modified organopolysiloxane according to the present invention may be a mixture of at least two types of sugar alcohol-modified organopolysiloxanes in which X′ in the aforementioned general formula (2′) consists of two types of xylitol residues represented by the structural formula: —C₃H₆—OCH₂[CH(OH)]₃CH₂OH and the structural formula: —C₃H₆—OCH{CH(OH)CH₂OH}₂ in a constitutional ratio (weight ratio) preferably ranging from 5:5 to 10:0 and in particular, preferably ranging from 8:2 to 10:0, in which the constitutional ratio is different from each other.

The divalent hydrocarbon group having 3 to 5 carbon atoms as R⁴ and the monovalent hydrocarbon group having 1 to 8 carbon atoms as R⁵ are the same as described above.

The blending amount of the aforementioned sugar alcohol-modified organopolysiloxane in the powder treatment agent of the present invention is not particularly restricted as long as effects of treating powders are exhibited, and can range, for example, from 50 to 100% by weight (mass), preferably ranges from 70 to 100% by weight (mass), and more preferably ranges from 90 to 100% by weight (mass) with respect to the amount of the powder treatment agent.

The aforementioned sugar alcohol-modified organopolysiloxane can provide the appropriate water repellency by orienting on the surface of the various powders. For this reason, the sugar alcohol-modified organopolysiloxane can be preferably used as the powder-surface treatment agent for the surface treatment of powders of cosmetics. The blending amount of the aforementioned sugar alcohol-modified organopolysiloxane in the powder-surface treatment agent of the present invention is not particularly restricted as long as effects of treating the surface of powders are exhibited, and can range, for example, from 50 to 100% by weight (mass), preferably ranges from 70 to 100% by weight (mass), and more preferably ranges from 90 to 100% by weight (mass) with respect to the amount of the powder-surface treatment agent.

The aforementioned sugar alcohol-modified organopolysiloxane of the present invention can exhibit good compatibility with various other components which are hydrophilic or hydrophobic in a cosmetic, and can improve dispersing properties and stability of the aforementioned powder(s) in a cosmetic containing powder(s). Therefore, the powder treatment agent and the powder-surface treatment agent of the present invention can improve the uniform dispersion properties of the aforementioned powder(s). In addition, a cosmetic containing powder(s) surface-treated with the aforementioned powder-surface treatment agent can exhibit increased stability and the aforementioned powder(s) can be uniformly dispersed in the cosmetic.

In the case of using the aforementioned sugar alcohol-modified organopolysiloxane in a surface treatment of powder(s), the aforementioned sugar alcohol-modified organopolysiloxane may preferably be used in an amount ranging from 0.1 to 10 parts by weight (mass) with respect to 100 parts by weight (mass) of the powder(s). If the amount is below the aforementioned lower limit, the effects obtained by the surface treatment may not sufficiently be exhibited. On the other hand, if the amount exceeds the aforementioned upper limit, remarkable change of the texture in accordance with the increased amount may not be obtained, and a tendency of obtaining a uniform mixture of the powders and the sugar alcohol-modified organopolysiloxane may be increased.

In addition, powder(s) may be subjected to a surface treatment by combining with other known surface treatments. As examples of other surface treatments, mention may be made of, for example, treatments with a methylhydrogenpolysiloxane, a silicone resin, a metal soap, a silane coupling agent, an inorganic oxide such as silica, alumina, titanium oxide or the like, or a fluorine compound such as a perfluoroalkylsilane, a perfluoroalkylphosphoric ester or the like. Therefore, the powder-surface treatment agent of the present invention may include other surface treatment agent(s) in an amount, for example, ranging from 0.1 to 50% by weight (mass), preferably ranging from 1 to 30% by weight (mass), and more preferably ranging from 5 to 10% by weight (mass).

The aforementioned sugar alcohol-modified organopolysiloxane of the present invention can be obtained by addition-reacting a mono-unsaturated ether compound of a sugar alcohol having a carbon-carbon double bond in a molecule with respect to a polyorganopolysiloxane having a reactive functional group, and in particular, an organopolysiloxane having a silicon-hydrogen bond.

The type of the addition reaction is not particularly restricted. The addition reaction can preferably be carried out in the presence of a hydrosilylation catalyst in view of controlling the reaction, purification and yield.

More particularly, the aforementioned sugar alcohol-modified organopolysiloxane of the present invention can be obtained as a product of a hydrosilylation reaction between a SiH group-containing siloxane and a mono-unsaturated ether compound of a sugar alcohol. Thereby, the sugar alcohol-containing organic group can be introduced into the polysiloxane chain.

For example, the aforementioned sugar alcohol-modified organopolysiloxane can be obtained by addition-reacting, in the presence of a hydrosilylation reaction catalyst, an organohydrogenpolysiloxane represented by the following general formula (2″):

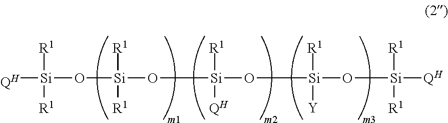

wherein
R¹ represents a monovalent organic group;
Y represents a polyoxyalkylene group represented by the following formula: —R⁴O(AO)ₙR⁵, wherein AO represents an oxyalkylene group having 2 to 4 carbon atoms; R⁴ represents a substituted or non-substituted, and linear or branched divalent hydrocarbon group having 3 to 5 carbon atoms; R⁵ represents a hydrogen atom, a substituted or non-substituted, and linear or branched monovalent hydrocarbon group having 1 to 8 carbon atoms, or a substituted or non-substituted, and linear or branched acyl group having 2 to 8 carbon atoms; and n=1 to 100;
Q^{H} represents any one of the aforementioned R¹, a hydrogen atom and a group represented by the aforementioned Y;
m1=0 to 700, preferably 0 to 500, and more preferably 0 250; m2=0 to 100, and preferably 0 to 50; and m3=0 to 50, and preferably 0 to 25, with the proviso that in the case of m2=0, at least one Q^{H} is a hydrogen atom,
with a mono-unsaturated ether compound of a sugar alcohol represented by the following general formula (1″-1):

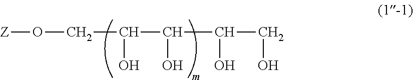

wherein Z represents an unsaturated organic group and preferably represents a substituted or non-substituted, and linear or branched unsaturated hydrocarbon group having 3 to 5 carbon atoms;
m is 1 or 2, and preferably 1,
or a mono-unsaturated ether compound of a sugar alcohol represented by the following general formula (1″-2):

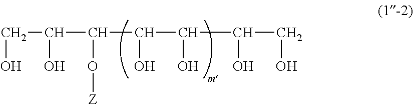

wherein Z is the same as defined above;
m′ is 0 or 1, and preferably 0,
in an amount of one or more molar equivalent thereof with respect to the hydrogen atom binding to the silicon atom in the organohydrogenpolysiloxane.

The unsaturated organic group is not particularly restricted as long as the organic group has an unsaturated group. A substituted or non-substituted, and linear or branched, unsaturated hydrocarbon group having 3 to 5 carbon atoms is preferred. As examples of unsaturated hydrocarbon groups having 3 to 5 carbon atoms, mention may be made of alkenyl groups such as a vinyl group, an allyl group, a butenyl group and the like. An allyl group is preferred.

As the aforementioned mono-unsaturated ether compound of a sugar alcohol, a monoallyl ether of a sugar alcohol is preferred, and a xylitol monoallyl ether (hereinafter, referred to as “xylitol monoallyl ether”) represented by the following structural formula: CH₂═CH—CH₂—OCH₂[CH(OH)]₃CH₂OH or represented by the following structural formula: CH₂═CH—CH₂—OCH{CH(OH)CH₂OH}₂ is more preferred. The xylitol monoallyl ether can be synthesized in accordance with a conventional method, and some products are commercially available.

As the aforementioned xylitol monoallyl ether, which is a raw material of the sugar alcohol-modified organopolysiloxane according to the present invention, either one or a mixture of a compound represented by the following structural formula: CH₂═CH—CH₂—OCH₂[CH(OH)]₃CH₂OH and a compound represented by the following structural formula: CH₂═CH—CH₂—OCH{CH(OH)CH₂OH}₂ can be used without particular restriction. Preferably, either one of the xylitol monoallyl ethers represented by the following structural formula: CH₂═CH—CH₂—OCH₂[CH(OH)]₃CH₂OH and represented by the following structural formula: CH₂═CH—CH₂—OC{CH(OH)CH₂OH}₂ is purified and used as a raw material. Alternatively, a xylitol monoallyl ether mixture containing xylitol monoallyl ethers represented by the following structural formula: CH₂═CH—CH₂—OCH₂[CH(OH)]₃CH₂OH and represented by the following structural formula: CH₂═CH—CH₂—OCH{CH(OH)CH₂OH}₂ in a weight (mass) ratio ranging from 5:5 to 10:0 can preferably be used as a raw material. In the latter case, use of the xylitol monoallyl ether having a ratio ranging from 8:2 to 10:0 is more preferred. In the case of using a ratio of 10:0, the raw material is a purified product consisting substantially of the xylitol monoallyl ether represented by the following structural formula: CH₂═CH—CH₂—OCH₂[CH(OH)]₃CH₂OH.

In addition, in order to obtain the aforementioned sugar alcohol-modified organopolysiloxane, a derivative of a sugar alcohol compound (a ketal compound), in which a hydroxyl group of the sugar alcohol compound corresponding to a sugar alcohol-modified group to be introduced is protected by a ketalizing agent such as 2,2-dimethoxypropane or the like in the presence of an acid catalyst, can also be used as a raw material. More particularly, a ketal derivative of the sugar alcohol having a carbon-carbon double bond in the molecule, which is obtained by purifying a reaction product between the aforementioned ketal compound and an alkenyl halide, is subjected to an addition reaction with an organopolysiloxane having silicon-hydrogen bonds. After the addition reaction, a de-ketalization reaction can be carried out by means of an acid hydrolysis treatment to deprotect the hydroxyl group. Thereby, the aforementioned sugar alcohol-modified organopolysiloxane according to the present invention can also be produced. Even by the aforementioned method using the aforementioned ketal derivative, after deprotection, the sugar alcohol-modified organopolysiloxane can be obtained. For this reason, any one of the preparation methods may be selected in accordance with the desirable yield or the conditions such as production facilities, purification of raw materials and the like. In addition, in order to improve a quality such as purity or a desirable property of the sugar alcohol-modified organopolysiloxane according to the present invention, any one of the preparation methods may be selected.

In order to improve the miscibility of the components in a cosmetic and improve feeling on touch thereof, during, before or after the aforementioned reaction, a different type of functional group may be introduced in an organopolysiloxane having a silicon-hydrogen bond by means of an addition reaction, and thereby, co-modification can be carried out. This is preferred. For example, the aforementioned co-modified product can be suitably produced by reacting together with an organohydrogensiloxane having silicon-binding hydrogen atoms at the side chain(s) and/or at the terminal(s) under the condition in at least which a sugar alcohol monoallyl ether having a carbon-carbon double bond in a molecule and a precursor compound of a functional group having one reactive unsaturated group in one molecule are co-present.

The hydrosilylation reaction is preferably carried out in the presence of a catalyst. As examples of the catalyst, mention may be made of a compound such as platinum, ruthenium, rhodium, palladium, osmium, iridium or the like. A platinum compound is, in particular, effective since the catalytic activity thereof is high. As examples of platinum compounds, mention may be made of chloroplatinic acid; platinum metal; a platinum metal-supported carrier such as platinum-supported alumina, platinum-supported silica, platinum-supported carbon black or the like; and a platinum complex such as platinum-vinylsiloxane complex, platinum phosphine complex, platinum-phosphite complex, platinum alcholate catalyst or the like. The usage amount of the catalyst may range from 0.5 to 1,000 ppm as a platinum metal in the case of using a platinum catalyst.

In addition, the aforementioned sugar alcohol-modified organopolysiloxane may be subjected to a hydrogenation treatment in order to ameliorate odor after the reaction due to the residual unsaturated compound. For the hydrogenation treatment, there are a method using a pressurized hydrogen gas and a method using a hydrogen adding agent such as a metal hydride or the like. In addition, in the aforementioned hydrogenation treatment, there are a homogeneous reaction and a heterogeneous reaction. One of these reactions can also be carried out, or the reactions can also be carried out in combination. Considering an advantage in that the used catalyst does not remain in a product, a heterogeneous catalytic hydrogenation reaction using a solid catalyst is most preferable.

As the solid catalyst (hydrogenation catalyst), a common noble metal-based catalyst such as a platinum-based catalyst, a palladium-based catalyst or the like, and a nickel-based catalyst can be used. More particular, as examples thereof, mention may be made of an elemental substance such as nickel, palladium, platinum, rhodium, cobalt or the like, and a catalyst of a combination of plural metals such as platinum-palladium, nickel-copper-chromium, nickel-copper-zinc, nickel-tungsten, nickel-molybdenum or the like. As examples of a catalyst carrier optionally used, mention may be made of activated carbon, silica, silica alumina, alumina, zeolite and the like. In addition, a copper-containing hydrogenation catalyst such as Cu—Cr, Cu—Zn, Cu—Si, Cu—Fe—Al, Cu—Zn—Ti and the like may be mentioned. The form of the aforementioned hydrogenation catalyst cannot be completely determined since the form may vary depending on the type of reactor, and can be appropriately selected from powders, granules, tablets and the like. In addition, the platinum catalyst used in the synthesis step (hydrosilylation reaction) can also be used as it is. The aforementioned hydrogenation catalyst can be used alone or in combination with two or more types thereof.

The hydrogenation treatment can also be used in order to purify a crude product of the sugar alcohol-modified organopolysiloxane obtained by the aforementioned addition reaction. More particularly, the aforementioned purification can be carried out by deodoration due to the hydrogenation treatment in a solvent or without a solvent in the presence of a hydrogenation catalyst. The aforementioned purified product can preferably be used in a cosmetic in which reduction of odor and miscibility with other cosmetic components are desired. In addition, as the pre-step or post-step of the aforementioned deodoration, a stripping treatment in which light products are removed by distillation by contacting a nitrogen gas with respect to a crude product or a hydrogenated product of a sugar alcohol-modified organopolysiloxane can preferably be carried out.

Alternatively, the odor of the crude product of the sugar alcohol-modified organopolysiloxane obtained by the aforementioned addition reaction can also be easily reduced by carrying out a stripping step in which light products are removed by distillation by contacting a nitrogen gas after an unreacted unsaturated product is hydrolyzed by adding an acid substance.

In the aforementioned hydrogenation treatment, acid treatment, and stripping treatment, solvents, reaction conditions, pressure-reduction conditions and the like used in purification of conventional organopolysiloxane copolymers or polyether-modified silicones can be applied and selected without any restrictions.

In order to adjust the dispersing properties and viscosity as a powder treatment agent and/or a powder-surface treatment agent, two or more types of the aforementioned sugar alcohol-modified organopolysiloxanes having different modification indexes, having different lengths of the polysiloxane chain, or having different binding sites (side chain/terminal) can be used in different rates together therewith.

The powder treatment agent comprising the aforementioned sugar alcohol-modified organopolysiloxane is useful as a raw material of a cosmetic, and powder which has been subjected to a surface treatment with the powder-surface treatment agent comprising the aforementioned sugar alcohol-modified organopolysiloxane are also useful as a raw material of a cosmetic. A mixture of a powder treatment agent comprising the aforementioned sugar alcohol-modified organopolysiloxane and powder is also useful as a cosmetic raw material. In addition, a composition which includes a powder treatment agent comprising the aforementioned sugar alcohol-modified organopolysiloxane, a powder or powders, and an oil agent or agents is, in particular, useful as a raw material of a cosmetic.

Hereinafter, the above aspects of the present invention are described in detail.

Powder

“Powder” in the present invention is that commonly used as a component of a cosmetic, and includes white and colored pigments and extender pigments. The white and colored pigments may be used in coloring a cosmetic, and on the other hand, the extender pigments may be used in improvement of feeling on touch of a cosmetic or the like. As the “powder” in the present invention, white or colored pigments and extender pigments which are commonly used in cosmetics can be used without any restrictions. One type of powder may be used, or two or more types of powders may preferably be blended.

With respect to powders, there is no restriction on the form thereof (sphere, bar, needle, plate, amorphous, spindle or the like), the particle size (aerosol, microparticle, pigment-grade particle, or the like), and the particle structure (porous, non-porous or the like) thereof. The average primary particle size of the powders preferably ranges from 1 nm to 100 μm.

As examples of powders, mention may be made of, for example, inorganic powders, organic powders, surfactant metal salt powders (metallic soaps), colored pigments, pearl pigments, metal powder pigments and the like. In addition, hybrid products of the aforementioned pigments can also be used.

More particularly, as examples of inorganic powders, mention may be made of titanium oxide, zirconium oxide, zinc oxide, cerium oxide, magnesium oxide, barium sulfate, calcium sulfate, magnesium sulfate, calcium carbonate, magnesium carbonate, talc, mica, kaolin, sericite, white mica, synthetic mica, phlogopite, lepidolite, black mica, lithia mica, silicic acid, silicic acid anhydride, aluminum silicate, sodium silicate, magnesium sodium silicate, magnesium silicate, aluminum magnesium silicate, calcium silicate, barium silicate, strontium silicate, metal salts of tungstic acid, hydroxyapatite, vermiculite, higilite, bentonite, montmorillonite, hectorite, zeolite, ceramic powder, dicalcium phosphate, alumina, aluminum hydroxide, boron nitride, and the like. As examples of organic powders, mention may be made of polyamide powder, polyester powder, polyethylene powder, polypropylene powder, polystyrene powder, polyurethane powder, benzoguanamine powder, polymethylbenzoguanamine powder, polytetrafluoroethylene powder, poly(methyl methacrylate) powder, cellulose, silk powder, nylon powder, nylon 12, nylon 6, silicone powder, polymethylsilsesquioxane spherical powder, copolymers of styrene and acrylic acid, copolymers of divinylbenzene and styrene, vinyl resin, urea resin, phenol resin, fluorine resin, silicone resin, acrylic resin, melamine resin, epoxy resin, polycarbonate resin, microcrystalline fiber powder, starch powder, lauroyl lysine and the like.

As examples of surfactant metal salt powders, mention may be made of zinc stearate, aluminum stearate, calcium stearate, magnesium stearate, zinc myristate, magnesium myristate, zinc palmitate, zinc laurate, zinc cetylphosphate, calcium cetylphosphate, sodium zinc cetylphosphate, and the like.

As examples of colored pigments, mention may be made of inorganic red pigments such as red iron oxide, iron oxide, iron hydroxide, iron titanate and the like; inorganic brown pigments such as gamma-iron oxide and the like; inorganic yellow pigments such as yellow iron oxide, ocher, and the like; inorganic black iron pigments such as black iron oxide, carbon black and the like; inorganic purple pigments such as manganese violet, cobalt violet, and the like; inorganic green pigments such as chromium hydroxide, chromium oxide, cobalt oxide, cobalt titanate, and the like; inorganic blue pigments such as Prussian blue, ultramarine blue, and the like; laked pigments of tar pigments such as Red No. 3, Red No. 104, Red No. 106, Red No. 201, Red No. 202, Red No. 204, Red No. 205, Red No. 220, Red No. 226, Red No. 227, Red No. 228, Red No. 230, Red No. 401, Red No. 505, Yellow No. 4, Yellow No. 5, Yellow No. 202, Yellow No. 203, Yellow No. 204, Yellow No. 401, Blue No. 1, Blue No. 2, Blue No. 201, Blue No. 404, Green No. 3, Green No. 201, Green No. 204, Green No. 205, Orange No. 201, Orange No. 203, Orange No. 204, Orange No. 206, Orange No. 207 and the like, laked pigments of natural pigments such as carminic acid, laccaic acid, carthamin, brazilin, crocin and the like.

As examples of pearl pigments, mention may be made of titanium oxide-coated mica, titanium mica, iron oxide-coated titanium mica, titanium oxide-coated mica, bismuth oxychloride, titanium oxide-coated bismuth oxychloride, titanium oxide-coated talc, fish scale foil, titanium oxide-coated colored mica, and the like.

As examples of metal powder pigments, mention may be made of powders of metals such as aluminum, gold, silver, copper, platinum, stainless steel, and the like.

In addition, in the aforementioned powders, a part or all parts thereof may, in particular, preferably be subjected to a surface treatment such as a water-repellent treatment, a hydrophilic treatment or the like. In addition, composited products in which the aforementioned powders are mutually composited may be used. In addition, surface-treated products in which the aforementioned powders have been subjected to a surface treatment with a general oil agent, a silicone compound other than the sugar alcohol-modified organopolysiloxane of the present invention, a fluorine compound, a surfactant, a thickening agent or the like can also be used. One type thereof or two or more types thereof can be used, as necessary.

The water-repellant treatments are not particularly restricted. The aforementioned powders can be treated with various types of water-repellant surface treatment agents. As examples thereof, mention may be made of organosiloxane treatments such as a methylhydrogenpolysiloxane treatment, a silicone resin treatment, a silicone gum treatment, an acryl silicone treatment, a fluorinated silicone treatment and the like; metallic soap treatments such as a zinc stearate treatment and the like; silane treatments such as a silane coupling agent treatment, an alkylsilane treatment and the like; fluorine compound treatments such as a perfluoroalkylsilane treatment, a perfluoroalkyl phosphate treatment, a perfluoro polyether treatment and the like; amino acid treatments such as an N-lauroyl-L-lysine treatment and the like; oil agent treatments such as a squalane treatment and the like; acryl treatments such as an alkyl acrylate treatment and the like. The aforementioned treatments can be used in combination of two or more types thereof.

As the powders, silicone elastomer powders can be used. The silicone elastomer powder is a crosslinked product of a linear diorganopolysiloxane mainly formed from a diorganosiloxane unit (D unit). The silicone elastomer powder can be preferably produced by crosslink-reacting an organohydrogenpolysiloxane having a silicon-binding hydrogen atom at the side chain or the terminal and a diorganopolysiloxane having an unsaturated hydrocarbon group such as an alkenyl group or the like at the side chain or the terminal, in the presence of a catalyst for a hydrosilylation reaction. The silicone elastomer powder has an increased flexibility and elasticity, and exhibits a superior oil-absorbing property, as compared with a silicone resin powder formed from T units and Q units. For this reason, the silicone elastomer powder absorbs sebum on the skin and can prevent makeup running. In addition, when a surface treatment is carried out by the aforementioned sugar alcohol-modified organopolysiloxane, a moisturized feeling on touch can be imparted without reducing a suede-like feeling on touch of the silicone elastomer powder. In addition, in the case of blending the aforementioned sugar alcohol-modified organopolysiloxane together with the silicone elastomer powder in a cosmetic, dispersion stability of the aforementioned powder in the entire cosmetic can be improved, and a stable cosmetic over time can be obtained.

The silicone elastomer powders can be in various forms such as a spherical form, a flat form, an amorphous form and the like. The silicone elastomer powders may be in the form of an oil dispersant. In the cosmetic of the present invention, silicone elastomer powders in the form of particles, which have a primary particle size observed by an electron microscope and/or an average primary particle size measured by a laser diffraction/scattering method ranging from 0.1 to 50 μm, and in which the primary particle is in a spherical form, can be preferably blended. In addition, the silicone elastomer constituting the silicone elastomer powders may have a hardness preferably not exceeding 80, and more preferably not exceeding 65, when measured by means of a type A durometer according to JIS K 6253 “Method for determining hardness of vulcanized rubber or thermoplastic rubber”.

The silicone elastomer powders may be subjected to a surface treatment with a silicone resin, silica or the like. As examples of the aforementioned surface treatments, mention may be made of, for example, those described in Japanese Unexamined Patent Application, First Publication No. H02-243612; Japanese Unexamined Patent Application, First Publication No. H08-12545; Japanese Unexamined Patent Application, First Publication No. H08-12546; Japanese Unexamined Patent Application, First Publication No. H08-12524; Japanese Unexamined Patent Application, First Publication No. H09-241511; Japanese Unexamined Patent Application, First Publication No. H10-36219; Japanese Unexamined Patent Application, First Publication No. H11-193331; Japanese Unexamined Patent Application, First Publication No. 2000-281523 and the like. As the silicone elastomer powders, crosslinking silicone powders listed in “Japanese Cosmetic Ingredients Codex (JCIC)” correspond thereto. As commercially available products of the silicone elastomer powders, there are Trefil E-506S, Trefil E-508, 9701 Cosmetic Powder, and 9702 Powder, manufactured by Dow Corning Toray Co., Ltd., and the like. As examples of the surface treatment agents, mention may be made of methylhydrogenpolysiloxane, silicone resins, metallic soap, silane coupling agents, inorganic oxides such as silica, titanium oxide and the like and fluorine compounds such as perfluoroalkylsilane, perfluoroalkyl phosphoric ester salts and the like.

A mixture of the aforementioned sugar alcohol-modified organopolysiloxane and powder(s) can be obtained by mixing an excess amount of the sugar alcohol-modified organopolysiloxane and powder(s). The aforementioned mixture is in the form of a powder dispersion in the sugar alcohol-modified organopolysiloxane. The blending amount of the powder(s) in the aforementioned mixture is not particularly restricted, and may preferably range from 50 to 99% by weight (mass) and more preferably may range from 80 to 90% by weight (mass) with respect to the total amount of the mixture.

The powder which has been subjected to a surface treatment with a powder surface treatment agent comprising the aforementioned sugar alcohol-modified organopolysiloxane, or a mixture of the aforementioned sugar alcohol-modified organopolysiloxane and powder(s) can provide powder(s) which can well be dispersed in a cosmetic. Therefore, they can be preferably used as a cosmetic raw material. In addition, a cosmetic obtained by using them as a raw material can exhibit superior stability.

The composition which contains a powder treatment agent comprising the aforementioned sugar alcohol-modified organopolysiloxane, powder(s) and oil agent(s) can also be used as a raw material of a cosmetic.

Oil Agent

The “oil agent” in the present invention is commonly used as a component of a cosmetic, and is not particularly restricted. The oil agent is usually in the form of a liquid at room temperature, and may be in the form of a solid such as a wax or in the form of a gum or a paste which has an increased viscosity and is thickened.

The oil agent is preferably at least one type of a liquid at 5 to 100° C., selected from the group consisting of a silicone oil, a non-polar organic compound and a low polar organic compound.

The silicone oils are hydrophobic, and the molecular structure thereof may be a cyclic, linear or branched structure. The viscosity of the silicone oils at 25° C. usually ranges from 0.65 to 100,000 mm²/s and preferably ranges from 0.65 to 10,000 mm²/s.

As examples of the aforementioned silicone oils, mention may be made of, for example, linear organopolysiloxanes, cyclic organopolysiloxanes, and branched organopolysiloxanes. Among these, volatile, linear organopolysiloxanes, cyclic organopolysiloxanes, and branched organopolysiloxanes are preferred.

As the linear organopolysiloxanes, cyclic organopolysiloxanes, and branched organopolysiloxanes, for example, organopolysiloxanes represented by the following general formulae: (3), (4) and (5):

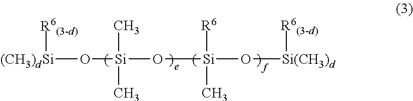

wherein
R⁶ is a hydrogen atom, a hydroxyl group or a group selected from monovalent non-substituted or fluorine- or amino-substituted, C₁₋₃₀ alkyl groups, aryl groups, alkoxy groups and groups represented by (CH₃)₃SiO{(CH₃)₂SiO}₁Si(CH₃)₂CH₂CH₂—, wherein
l is an integer ranging from 0 to 1,000;
d is an integer ranging from 0 to 3;
e is an integer ranging from 0 to 1,000; and
f is an integer ranging from 0 to 1,000, with the proviso that 1≦e+f≦2,000,

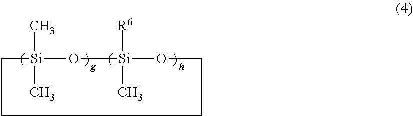

wherein
R⁶ is the same as defined above;
g is an integer ranging from 0 to 8; and
h is an integer ranging from 0 to 8, with the proviso that 3≦g+h≦8,

R⁶₍₄₋ᵢ₎Si(OSiCH₃)ⱼ (5)

wherein
R⁶ is the same as defined above;
i is an integer ranging from 1 to 4; and
j is an integer ranging from 0 to 500, can be used.

As examples of monovalent non-substituted or fluorine- or amino-substituted C₁₋₃₀ alkyl groups, aryl groups, and alkoxy groups having 1 to 30 carbon atoms, mention may be made of, for example, linear or branched alkyl groups having 1 to 30 carbon atoms such as a methyl group, an ethyl group, a propyl group, a butyl group, a pentyl group, a hexyl group, a heptyl group, an octyl group, a decyl group, a dodecyl group and the like; cycloalkyl groups having 3 to 30 carbon atoms such as a cylopentyl group, a cyclohexyl group and the like; aryl groups having 6 to 30 carbon atoms such as a phenyl group, a tolyl group, a xylyl group, a naphthyl group and the like; alkoxy groups having 1 to 30 carbon atoms such as a methoxy group, an ethoxy group, a propoxy group and the like; and substituted groups thereof, in which hydrogen atoms binding to carbon atoms of the aforementioned groups are at least partially substituted by a fluorine atom or an amino group. A non-substituted alkyl group or aryl group is preferred, and a non-substituted alkyl group or aryl group having 1 to 6 carbon atoms or aryl group is further preferred. A methyl group, an ethyl group or a phenyl group is, in particular, preferred.

As examples of linear organopolysiloxanes, mention may be made of a dimethylpolysiloxane in which both molecular terminals are capped with trimethylsiloxy groups (dimethylsilicone with a low viscosity such as 2 mPa·s or 6 mPa·s to dimethylsilicone with a high viscosity such as 1,000,000 mPa·s), an organohydrogenpolysiloxane, a methylphenylpolysiloxane in which both molecular terminals are capped with trimethylsiloxy groups, a copolymer of methylphenylsiloxane and dimethylsiloxane in which both molecular terminals are capped with trimethylsiloxy groups, a diphenylpolysiloxane in which both molecular terminals are capped with trimethylsiloxy groups, a copolymer of diphenylsiloxane and dimethylsiloxane in which both molecular terminals are capped with trimethylsiloxy groups, a trimethylpentaphenyltrisiloxane, a phenyl(trimethylsiloxy)siloxane, a methylalkylpolysiloxane in which both molecular terminals are capped with trimethylsiloxy groups, a copolymer of methylalkylsiloxane and dimethylpolysiloxane in which both molecular terminals are capped with trimethylsiloxy groups, a copolymer of methyl(3,3,3-trifluoropropyl)siloxane and dimethylsiloxane in which both molecular terminals are capped with trimethylsiloxy groups, an α,ω-dihydroxypolydimethylsiloxane, an α,ω-diethoxypolydimethylsiloxane, a 1,1,1,3,5,5,5-heptamethyl-3-octyltrisiloxane, a 1,1,1,3,5,5,5-heptamethyl-3-dodecyltrisiloxane, a 1,1,1,3,5,5,5-heptamethyl-3-hexadecyltrisiloxane, a tristrimethylsiloxymethylsilane, a tristrimethylsiloxyalkylsilane, a tetrakistrimethylsiloxysilane, tetramethyl-1,3-dihydroxydisiloxane, an octamethyl-1,7-dihydroxytetrasiloxane, a hexamethyl-1,5-diethoxytrisiloxane, a hexamethyldisiloxane, an octamethyltrisiloxane, a higher alkoxy-modified silicone, a higher fatty acid-modified silicone, dimethiconol and the like.

More particularly, as examples of cyclic organopolysiloxanes, mention may be made of hexamethylcyclotrisiloxane (D3), octamethylcyclotetrasiloxane (D4), decamethylcyclopentasiloxane (D5), dodecamethylcyclohexasiloxane (D6), 1,1-diethylhexamethylcyclotetrasiloxane, phenylheptamethylcyclotetrasiloxane, 1,1-diphenylhexamethylcyclotetrasiloxane, 1,3,5,7-tetravinyltetramethylcyclotetrasiloxane, 1,3,5,7-tetramethylcyclotetrasiloxane, 1,3,5,7-tetracyclohexyltetramethylcyclotetrasiloxane, tris(3,3,3-trifluoropropyl)trimethylcyclotrisiloxane, 1,3,5,7-tetra(3-methacryloxypropyl)tetramethylcyclotetrasiloxane, 1,3,5,7-tetra(3-acryloxypropyl)tetramethylcyclotetrasiloxane, 1,3,5,7-tetra(3-carboxypropyl)tetramethylcyclotetrasiloxane, 1,3,5,7-tetra(3-vinyloxypropyl)tetramethylcyclotetrasiloxane, 1,3,5,7-tetra(p-vinylphenyl)tetramethylcyclotetrasiloxane, 1,3,5,7-tetra[3-(p-vinylphenyl)propyl]tetramethylcyclotetrasiloxane, 1,3,5,7-tetra(N-acryloyl-N-methyl-3-aminopropyl)tetramethylcyclotetrasiloxane, 1,3,5,7-tetra(N,N-bis(lauroyl)-3-aminopropyl)tetramethylcyclotetrasiloxane and the like.

As examples of branched organopolysiloxanes, mention may be made of methyltristrimethylsiloxysilane, ethyltristrimethylsiloxysilane, propyltristrimethylsiloxysilane, tetrakistrimethylsiloxysilane, phenyltristrimethylsiloxysilane and the like.

As the non-polar organic compounds and the low polar organic compounds, a hydrocarbon oil and a fatty acid ester oil are preferred. They are widely used, in particular, as a base material of a makeup cosmetic. The sugar alcohol-modified organopolysiloxane of the present invention exhibits a superior dispersing property with respect to these non-silicone-based oil agents. For this reason, hydrocarbon oils and fatty acid ester oils can be stably blended in cosmetics, and a moisturizing property obtained by these non-silicone-based oil agents can be maintained. Therefore, the aforementioned sugar alcohol-modified organopolysiloxane can improve stability of the aforementioned non-silicone-based oil agents in cosmetics over time.

By using the aforementioned silicone oils in combination with the hydrocarbon oils and/or fatty acid ester oils, advantages can be obtained in that moisture on the skin can be maintained, and a moisturizing sensation (also referred to as “moisturizing feeling on touch”) such as moisturizing skin or hair and smooth feeling on touch, in addition to a refreshing feeling on touch which the silicone oils inherently possess, can be provided in cosmetics, and stability over time of cosmetics is not impaired. In addition, use of the cosmetics containing the aforementioned silicone oils in combination with the hydrocarbon oils and/or fatty acid ester oils provides advantages in that these moisturizing components (the hydrocarbon oils and/or fatty acid ester oils) can be stably and uniformly applied on the skin or hair, the moisturizing effects of the moisturizing components on the skin can be increased, and therefore, superior smoothness and a superior moisturizing feeling can be provided, as compared with cosmetics containing only non-silicone oils (the hydrocarbon oils and/or fatty acid ester oils).

As examples of hydrocarbon oils, mention may be made of liquid paraffin, light liquid isoparaffin, heavy liquid isoparaffin, vaseline, n-paraffin, isoparaffin, isododecane, isohexadecane, polyisobutylene, hydrogenated polyisobutylene, polybutene, ozokerite, ceresin, microcrystalline wax, paraffin wax, polyethylene wax, polyethylene/polypropylene wax, squalane, squalene, pristane, polyisoprene and the like.

As examples of fatty acid ester oils, mention may be made of hexyldecyl octanoate, cetyl octanoate, isopropyl myristate, isopropyl palmitate, butyl stearate, hexyl laurate, myristyl myristate, oleyl oleate, decyl oleate, octyldodecyl myristate, hexyldecyl dimethyloctanoate, cetyl lactate, myristyl lactate, diethyl phthalate, dibutyl phthalate, lanolin acetate, ethylene glycol monostearate, propylene glycol monostearate, propylene glycol dioleate, glyceryl monostearate, glyceryl monooleate, glyceryl tri-2-hexanoate, trimethylolpropane tri-2-ethylhexanoate, ditrimethylolpropane triethylhexanoate, ditrimethylolpropane isostearate/sebacate, trimethylolpropane trioctanoate, trimethylolpropane triisostearate, diisopropyl adipate, diisobutyl adipate, 2-hexyldecyl adipate, di-2-heptylundecyl adipate, diisostearyl malate, hydrogenated castor oil monoisostearate, N-alkylglycol monoisostearate, octyldodecyl isostearate, isopropyl isostearate, isocetyl isostearate, ethylene glycol di-2-ethylhexanoate, cetyl 2-ethylhexanoate, pentaerythritol tetra-2-ethylhexanoate, octyldodecyl gum ester, ethyl oleate, octyldodecyl oleate, neopentylglycol dicaprate, triethyl citrate, 2-ethylhexyl succinate, dioctyl succinate, isocetyl stearate, diisopropyl sebacate, di-2-ethylhexyl sebacate, diethyl sebacate, dioctyl sebacate, dibutyloctyl sebacate, cetyl palmitate, octyldodecyl palmitate, octyl palmitate, 2-ethylhexyl palmitate, 2-hexyldecyl palmitate, 2-heptylundecyl palmitate, cholesteryl 12-hydroxystearate, dipentaerythritol fatty acid ester, 2-hexyldecyl myristate, ethyl laurate, 2-octyldodecyl N-lauroyl-L-glutamate, di(cholesteryl/behenyl/octyldodecyl) N-lauroyl-L-glutamate, di(cholesteryl/octyldodecyl) N-lauroyl-L-glutamate, di(phytosteryl/behenyl/octyldodecyl) N-lauroyl-L-glutamate, di(phytosteryl/octyldodecyl) N-lauroyl-L-glutamate, isopropyl N-lauroylsarcosinate, diisostearyl malate, neopentylglycol dioctanoate, isodecyl neopentanoate, isotridecyl neopentanoate, isostearyl neopentanoate, isononyl isononanoate, isotridecyl isononanoate, octyl isononanoate, isotridecyl isononanoate, diethylpentanediol dineopentanoate, methylpentanediol dineopentanoate, octyldodecyl neodecanoate, 2-butyl-2-ethyl-1,3-propanediol dioctanoate, pentaerythrityl tetraoctanoate, pentaerythrityl hydrogenated rosin, pentaerythrityl triethylhexanoate, dipentaerythrityl (hydroxystearate/stearate/rosinate), polyglyceryl tetraisostearate, polyglyceryl-10 nonaisostearate, polyglyceryl-8 deca(erucate/isostearate/ricinoleate), (hexyldecanoic acid/sebacic acid) diglyceryl oligoester, glycol distearate (ethylene glycol distearate), diisopropyl dimmer dilinoleate, diisostearyl dimmer dilinoleate, di(isostearyl/phytosteryl) dimmer dilinoleate, (phytosteryl/behenyl) dimmer dilinoleate, (phytosteryl/isostearyl/cetyl/stearyl/behenyl) dimmer dilinoleate, dimmer dilinoleyl dimmer dilinoleate, dimmer dilinoleyl diisostearate, dimmer dilinoleyl hydrogenated rosin condensate, dimmer dilinoleic acid hardened castor oil, hydroxyalkyl dimmer dilinoleyl ether, glyceryl triisooctanoate, glyceryl triisostearate, glyceryl trimyristate, glyceryl triisopalmitate, glyceryl trioctanoate, glyceryl trioleate, glyceryl diisostearate, glyceryl tri(caprylate/caprate), glyceryl tri(caprylate/caprate/myristate/stearate), hydrogenated rosin triglyceride (hydrogenated ester gum), rosin triglyceride (ester gum), glyceryl behenate eicosane dioate, glyceryl di-2-heptylundecanoate, diglyceryl myristate isostearate, cholesteryl acetate, cholesteryl nonanoate, cholesteryl stearate, cholesteryl isostearate, cholesteryl oleate, cholesteryl 12-hydroxystearate, cholesteryl ester of macadamia nut oil fatty acid, phytosteryl ester of macadamia nut oil fatty acid, phytosteryl isostearate, cholesteryl ester of soft lanolin fatty acid, cholesteryl ester of hard lanolin fatty acid, cholesteryl ester of long-chain branched fatty acid, cholesteryl ester of long-chain α-hydroxy fatty acid, octyldodecyl ricinoleate, octyldodecyl ester of lanolin fatty acid, octyldodecyl erucate, isostearic acid hardened castor oil, ethyl ester of avocado fatty acid, isopropyl ester of lanolin fatty acid, and the like.

As the low polar organic compound, a higher alcohol having 10 to 30 carbon atoms can be used. The aforementioned higher alcohol is a saturated or unsaturated monovalent aliphatic alcohol, and the moiety of the hydrocarbon group thereof may be linear or branched, but a linear one is preferred. As examples of higher alcohols having 10 to 30 carbon atoms, mention may be made of lauryl alcohol, myristyl alcohol, palmityl alcohol, stearyl alcohol, behenyl alcohol, hexadecyl alcohol, oleyl alcohol, isostearyl alcohol, hexyldodecanol, octyldodecanol, cetostearyl alcohol, 2-decyltetradecinol, cholesterol, sitosterol, phytosterol, lanosterol, lanolin alcohol, hydrogenated lanolin alcohol and the like. In the present invention, use of a higher alcohol having a melting point ranging from 40 to 80° C. or use of a combination of plural higher alcohols so as to have a melting point thereof ranging from 40 to 70° C. is preferred.

The blending amount of the oil agent(s) as the cosmetic raw material in the aforementioned composition is not particularly restricted, and may preferably range from 0.1 to 50% by weight (mass) and more preferably may range from 0.5 to 25% by weight (mass) with respect to the amount of the cosmetic raw material.

The aforementioned cosmetic raw material(s) can be used by being blended in cosmetics.

The cosmetic of the present invention can include the aforementioned sugar alcohol-modified organopolysiloxane and the aforementioned powder(s), derived from the aforementioned cosmetic raw material(s), as essential components, and can exhibit a superior dispersing property of the aforementioned powder(s). Therefore, the cosmetic of the present invention can exhibit superior stability over time and has a superior sensation during use.

The blending amount of the powder(s) in the cosmetic of the present invention may preferably range from 0.1 to 99% by weight (mass) in the total amount of the cosmetic. In particular, the blending amount in the case of a powdery solid cosmetic may preferably range from 80 to 99% by weight (mass) in the total amount of the cosmetic since the powder(s) are used as the base material of the cosmetic.

The blending amount of the aforementioned sugar alcohol-modified organopolysiloxane in the cosmetic of the present invention may preferably range from 0.1 to 20% by weight (mass), and more preferably may range from 1 to 10% by weight (mass) with respect to the total amount of the cosmetic.

The cosmetic of the present invention may further contain an oil agent or agents in addition to the aforementioned raw material(s) for cosmetics. As the aforementioned oil agent(s), the same oil agent(s) as described above can be used.

The blending amount of the oil agent(s) in the cosmetic of the present invention can appropriately be selected in accordance with the formulation, type, application part, and important properties of the cosmetic, and may preferably range from 0.1 to 90% by weight (mass) and more preferably may range from 0.5 to 70% by weight (mass) with respect to the total amount of the cosmetic. In addition, two or more types of the oil agents with different viscosities can also preferably blended.

In particular, in the case of using no oil agent as a base material of a cosmetic, as the case of an oil-in-water emulsion cosmetic, a non-silicone-based oil agent may preferably be blended in an amount ranging from 0.1 to 50% by weight (mass) and more preferably ranging from 0.5 to 25% by weight (mass), with respect to the total amount of the cosmetic. Thereby, moisture-retaining properties, moisturized sensation and a smooth sensation during use can be provided. On the other hand, in the case of using an oil agent as a base material of a cosmetic, as the case of a water-in-oil emulsion cosmetic, the non-silicone-based oil agent may preferably be blended in an amount ranging from 50 to 95% by weight (mass) and more preferably ranging from 70 to 90% by weight (mass), with respect to the total amount of the cosmetic. Thereby, the stable formulations and outer appearances of the cosmetics can be maintained, and total compatibility with other oil-based raw materials can be improved.

In the cosmetics of the present invention, in addition to the aforementioned oil agents, fats and oils, higher fatty acids, fluorine-based oils and the like may be used as other oil agents, and they may be used in combination of two or more types thereof. In particular, fats and oils derived from vegetables provide a healthy image due to natural products and exhibit a superior moisture-retaining property and superior compatibility on the skin. For this reason, they can preferably be used in a cosmetic of the present invention. The aforementioned other oil agents may preferably be blended in the amount ranging from 0.5 to 25% by weight (mass) of the total amount of the cosmetic. Hereinafter, the oil agents other than the silicone oils, hydrocarbon oils, fatty acid ester oils, and higher alcohols, which can be used in the present invention, are described in detail.

As examples of natural animal or vegetable fats and oils and semi-synthetic fats and oils, mention may be made of avocado oil, linseed oil, almond oil, ibota wax, perilla oil, olive oil, cacao butter, kapok wax, kaya oil, carnauba wax, liver oil, candelilla wax, beef tallow, hydrogenated beef tallow, apricot kernel oil, spermaceti wax, hydrogenated oil, wheat germ oil, sesame oil, rice germ oil, rice bran oil, sugar cane wax, sasanqua oil, safflower oil, shear butter, Chinese tung oil, cinnamon oil, jojoba wax, olive oil, squalane, shellac wax, turtle oil, soybean oil, tea seed oil, camellia oil, evening primrose oil, corn oil, lard, rapeseed oil, Japanese tung oil, rice bran wax, germ oil, horse fat, persic oil, palm oil, palm kernel oil, castor oil, hydrogenated castor oil, castor oil fatty acid methyl ester, sunflower oil, grape oil, bayberry wax, jojoba oil, hydrogenated jojoba ester, macadamia nut oil, beeswax, mink oil, cottonseed oil, cotton wax, Japanese wax, Japanese wax kernel oil, montan wax, coconut oil, hydrogenated coconut oil, tri-coconut oil fatty acid glyceride, mutton tallow, peanut oil, lanolin, liquid lanolin, reduced lanolin, lanolin alcohol, hard lanolin, lanolin acetate, lanolin fatty acid isopropyl ester, POE lanolin alcohol ether, POE lanolin alcohol acetate, lanolin fatty acid polyethylene glycol, POE hydrogenated lanolin alcohol ether, POE cholesterol ether, monostearyl glycerol ether (batyl alcohol), monooleyl glycerol ether (selachyl alcohol), egg yolk oil and the like, with the proviso that POE means polyoxyethylene.

As examples of higher fatty acids, mention may be made of, for example, lauric acid, myristic acid, palmitic acid, stearic acid, behenic acid, undecylenic acid, oleic acid, linolic acid, linolenic acid, arachidonic acid, eicosapentaenoic acid (EPA), docosahexaenoic acid (DHA), isostearic acid, 12-hydroxystearic acid, and the like.

As examples of fluorine-based oils, mention may be made of perfluoro polyether, perfluorodecalin, perfluorooctane and the like.

In addition, the cosmetic of the present invention may contain water. Therefore, the cosmetic of the present invention can be in the form of an oil-in-water emulsion or a water-in-oil emulsion. In this case, the cosmetic of the present invention exhibits superior emulsion stability and a superior sensation during use.

Water is not particularly restricted as long as it does not include any harmful components for human bodies and is clean. As examples thereof, mention may be made of tap water, purified water, and mineral water. In addition, in the emulsion composition of the present invention, the blending amount of water preferably ranges from 2 to 98% by weight (mass), in the case in which the total amount of all components of the emulsion is 100% by weight (mass). In a cosmetic in the form of a gel emulsion, optional components which are water soluble may be previously blended in water.

The form of the emulsion may be not only an oil-in-water emulsion or water-in-oil emulsion, but also a multiple emulsion or microemulsion thereof. The form of the emulsion (oil-in-water type or water-in-oil type) and the particle size of the emulsion can be appropriately selected or adjusted.

In the case of the cosmetic of the present invention in the form of an oil-in-water emulsion, the dispersion phase of the aforementioned cosmetic is formed from particles of oil agents emulsified by the aforementioned sugar alcohol-modified organopolysiloxane. The average particle size thereof can be measured by a conventional measurement device using a laser diffraction/scattering method or the like. The cosmetic in the form of an oil-in-water emulsion may be a transparent microemulsion in which the average particle size of the dispersion phase measured is 0.1 nm or less, or may be a milky emulsion having a large particle size so that the average particle size exceeds 10.0 nm. In addition, in order to improve stability and transparency of the outer appearance of the emulsion, the emulsion particles can be miniaturized. In particular, in order to improve the adhesive property with respect to the hair or skin or sensation during use, an emulsion having an average particle size ranging from 0.5 to 20 nm can be selected, and is preferred.

The cosmetic in the form of an oil-in-water emulsion or a water-in-oil emulsion can be produced by mixing components of the aforementioned cosmetic using a mechanical force by means of an apparatus such as a homomixer, a paddle mixer, a Henschel mixer, a homodisper, a colloid mill, a propeller stirrer, a homogenizer, an in-line type continuous emulsifier, an ultrasonic emulsifier, a vacuum kneader or the like.

For example, the aforementioned sugar alcohol-modified organopolysiloxane is mixed together with the powders and the oil agent in the optional presence of an alcohol such as ethanol or the like, and thereby, a cosmetic raw material which is a premix of an emulsion of a uniform solubilizing product is formed. The aforementioned premix is mixed with water by means of the aforementioned apparatus, and thereby, a cosmetic in the form of a uniform oil-in-water emulsion or water-in-oil emulsion can be produced.

The aforementioned sugar alcohol-modified organopolysiloxane is, in particular, preferred as a raw material (surfactant) when preparing a cosmetic in the form of a water-in-oil emulsion.

The cosmetics of the present invention may comprise one or more types of other surfactants. The other surfactants can be selected from the group consisting of anionic surfactants, cationic surfactants, nonionic surfactants, amphoteric surfactants and semi-polar surfactants.

The blending amount of the other surfactants in the cosmetic of the present invention is not particularly restricted. In order to improve a cleansing property, the other surfactants can be blended in an amount ranging from 0.1 to 90% by weight (mass) in the total amount of the cosmetic. In view of a cleansing property, the amount is preferably 25% by weight (mass) or more.

As examples of anionic surfactants, mention may be made of saturated or unsaturated fatty acid salts such as sodium laurate, sodium stearate, sodium oleate, sodium linoleate and the like; alkylsulfuric acid salts; alkylbenzenesulfonic acids such as hexylbenzenesulfonic acid, octylbenzenesulfonic acid, dodecylbenzenesulfonic acid and the like, as well as salts thereof; polyoxyalkylene alkyl ether sulfuric acid salts; polyoxyalkylene alkenyl ether sulfuric acid salts; polyoxyethylene alkylsulfuric ester salts; sulfosuccinic acid alkyl ester salts; polyoxyalkylene sulfosuccinic acid alkyl ester salts; polyoxyalkylene alkylphenyl ether sulfuric acid salts; alkanesulfonic acid salts; octyltrimethylammonium hydroxide; dodecyltrimethylammonium hydroxide; alkyl sulfonates; polyoxyethylene alkylphenyl ether sulfuric acid salts; polyoxyalkylene alkyl ether acetic acid salts; alkyl phosphoric acid salts; polyoxyalkylene alkyl ether phosphoric acid salts; acylglutamic acid salts; α-acylsulfonic acid salts; alkylsulfonic acid salts; alkylallylsulfonic acid salts; α-olefinsulfonic acid salts; alkylnaphthalene sulfonic acid salts; alkanesulfonic acid salts; alkyl- or alkenylsulfuric acid salts; alkylamidesulfuric acid salts; alkyl- or alkenylphosphoric acid salts; alkylamidephosphoric acid salts; alkyloylalkyl taurine salts; N-acylamino acid salts; sulfosuccinic acid salts; alkyl ether carboxylic acid salts; amide ether carboxylic acid salts; α-sulfofatty acid ester salts; alanine derivatives; glycine derivatives; and arginine derivatives. As examples of salts, mention may be made of alkali metal salts such as a sodium salt and the like, alkaline earth metal salts such as a magnesium salt and the like, alkanolamine salts such as a triethanolamine salt and the like, and an ammonium salt.

As examples of cationic surfactants, mention may be made of alkyltrimethylammonium chloride, stearyltrimethylammonium chloride, lauryltrimethylammonium chloride, cetyltrimethylammonium chloride, beef tallow alkyltrimethylammonium chloride, behenyltrimethylammonium chloride, stearyltrimethylammonium bromide, behenyltrimethylammonium bromide, distearyldimethylammonium chloride, dicocoyldimethylammonium chloride, dioctyldimethylammonium chloride, di(POE) oleylmethylammonium (2 EO) chloride, benzalkonium chloride, alkyl benzalkonium chloride, alkyl dimethylbenzalkonium chloride, benzethonium chloride, stearyl dimethylbenzylammonium chloride, lanolin derivative quaternary ammonium salt, stearic acid diethylaminoethylamide, stearic dimethylaminopropylamide, behenic acid amide propyldimethyl hydroxypropylammonium chloride, stearoyl colaminoformyl methylpyridinium chloride, cetylpyridinium chloride, tall oil alkylbenzyl hydroxyethylimidazolinium chloride, and benzylammonium salt.

As examples of nonionic surfactants, mention may be made of polyoxyalkylene ethers, polyoxyalkylene alkyl ethers, polyoxyalkylene fatty acid esters, polyoxyalkylene fatty acid diesters, polyoxyalkylene resin acid esters, polyoxyalkylene (hardened) castor oils, polyoxyalkylene alkyl phenols, polyoxyalkylene alkyl phenyl ethers, polyoxyalkylene phenyl phenyl ethers, polyoxyalkylene alkyl esters, polyoxyalkylene alkyl esters, sorbitan fatty acid esters, polyoxyalkylene sorbitan alkyl esters, polyoxyalkylene sorbitan fatty acid esters, polyoxyalkylene sorbitol fatty acid esters, polyoxyalkylene glycerol fatty acid esters, polyglycerol alkyl ethers, polyglycerol fatty acid esters, sucrose fatty acid esters, fatty acid alkanolamides, alkylglucosides, polyoxyalkylene fatty acid bisphenyl ethers, polypropylene glycol, diethylene glycol, polyoxyalkylene-modified silicones, polyglyceryl-modified silicones, glyceryl-modified silicones, sugar-modified silicones, fluorine-based surfactants, polyoxyethylene/polyoxypropylene block polymers, and alkyl polyoxyethylene/polyoxypropylene block polymer ethers. A polyoxyalkylene-modified silicone, a polyglycerol-modified silicone, or a glycerol-modified silicone in which an alkyl branch, a linear silicone branch, a siloxane dendrimer branch or the like may be possessed together with a hydrophilic group at the same time, if necessary, can also be preferably used.

The sugar alcohol-modified organopolysiloxane of the present invention possesses a hydrophilic moiety and a hydrophobic moiety in a molecule, and for this reason, the sugar alcohol-modified organopolysiloxane has a function as a dispersant. For this reason, in the case of using the sugar alcohol-modified organopolysiloxane together with a silicone-based nonionic surfactant, the organopolysiloxane may function as an auxiliary agent for improving stability of the nonionic surfactant and improve stability of the entire preparation. In particular, the aforementioned sugar alcohol-modified organopolysiloxane is preferably used together with a polyoxyalkylene-modified silicone, a polyglycerol-modified silicone, and a glycerol-modified silicone.

As examples of amphoteric surfactants, mention may be made of imidazoline-type, amidobetaine-type, alkylbetaine-type, alkylamidobetaine-type, alkylsulfobetaine-type, amidosulfobetaine-type, hydroxysulfobetaine-type, carbobetaine-type, phosphobetaine-type, aminocarboxylic acid-type, and amidoamino acid-type amphoteric surfactants. More particularly, as examples thereof, mention may be made of imidazoline-type amphoteric surfactants such as sodium 2-undecyl-N,N,N-(hydroxyethylcarboxymethyl)-2-imidazoline, 2-cocoyl-2-imidazolinium hydroxide-1-carboxyethyloxy disodium salt and the like; alkylbetaine-type amphoteric surfactants such as lauryl dimethylaminoacetic acid betaine, myristyl betaine and the like; and amidobetaine-type amphoteric surfactants such as coconut oil fatty acid amidopropyl dimethylamino acetic acid betaine, palm kernel oil fatty acid amidopropyl dimethylamino acetic acid betaine, beef tallow fatty acid amidopropyl dimethylamino acetic acid betaine, hardened beef tallow fatty acid amidopropyl dimethylamino acetic acid betaine, lauric amidopropyl dimethylamino acetic acid betaine, myristic amidopropyl dimethylamino acetic acid betaine, palmitic amidopropyl dimethylamino acetic acid betaine, stearic amidopropyl dimethylamino acetic acid betaine, oleic amidopropyl dimethylamino acetic acid betaine and the like; alkyl sulfobetaine-type amphoteric surfactants such as coconut oil fatty acid dimethyl sulfopropyl betaine and the like; alkylhydroxy sulfobetaine-type amphoteric surfactants such as lauryl dimethylaminohydroxy sulfobetaine and the like; phosphobetaine-type amphoteric surfactants such as laurylhydroxy phosphobetaine and the like; amidoamino acid-type amphoteric surfactants such as sodium N-lauroyl-N′-hydroxyethyl-N′-carboxymethyl ethylenediamine, sodium N-oleoyl-N′-hydroxyethyl-N′-carboxymethyl ethylenediamine, sodium N-cocoyl-N′-hydroxyethyl-N′-carboxymethyl ethylenediamine, potassium N-lauroyl-N′-hydroxyethyl-N′-carboxymethyl ethylenediamine, potassium N-oleoyl-N′-hydroxyethyl-N′-carboxymethyl ethylenediamine, sodium N-lauroyl-N-hydroxyethyl-N′-carboxymethyl ethylenediamine, sodium N-oleoyl-N-hydroxyethyl-N′-carboxymethyl ethylenediamine, sodium N-cocoyl-N-hydroxyethyl-N′-carboxymethyl ethylenediamine, monosodium N-lauroyl-N-hydroxyethyl-N′,N′-dicarboxymethyl ethylenediamine, monosodium N-oleoyl-N-hydroxyethyl-N′,N′-dicarboxymethyl ethylenediamine, monosodium N-cocoyl-N-hydroxyethyl-N′,N′-dicarboxymethyl ethylenediamine, disodium N-lauroyl-N-hydroxyethyl-N′,N′-dicarboxymethyl ethylenediamine, disodium N-oleoyl-N-hydroxyethyl-N′,N′-dicarboxymethyl ethylenediamine, disodium N-cocoyl-N-hydroxyethyl-N′,N′-dicarboxymethyl ethylenediamine and the like.

As examples of semi-polar surfactants, mention may be made of alkylamine oxide-type surfactants, alkylamine oxides, alkylamide amine oxides, alkylhydroxyamine oxides and the like. Alkyldimethylamine oxides having 10 to 18 carbon atoms, alkoxyethyl dihydroxyethylamine oxides having 8 to 18 carbon atoms and the like are preferably used. More particularly, as examples thereof, mention may be made of dodecyldimethylamine oxide, dimethyloctylamine oxide, diethyldecylamine oxide, bis-(2-hydroxyethyl)dodecylamine oxide, dipropyltetradecylamine oxide, methylethylhexadecylamine oxide, dodecylamidopropyldimethylamine oxide, cetyldimethylamine oxide, stearyldimethylamine oxide, tallow dimethylamine oxide, dimethyl-2-hydroxyoctadecylamine oxide, lauryldimethylamine oxide, myristyldimethylamine oxide, stearyldimethylamine oxide, isostearyldimethylamine oxide, coconut fatty acid alkyldimethylamine oxide, caprylic amide propyldimethylamine oxide, capric amide propyldimethylamine oxide, lauric amide propyldimethylamine oxide, myristic amide propyldimethylamine oxide, palmitic amide propyldimethylamine oxide, stearic amide propyldimethylamine oxide, isostearic amide propyldimethylamine oxide, oleic amide propyldimethylamine oxide, ricinoleic amide propyldimethylamine oxide, 12-hydroxystearic amide propyldimethylamine oxide, coconut fatty acid amide propyldimethylamine oxide, palm kernel oil fatty acid amide propyldimethylamine oxide, castor oil fatty acid amide propyldimethylamine oxide, lauric amide ethyldimethylamine oxide, myristic amide ethyldimethylamine oxide, coconut fatty acid amide ethyldimethylamine oxide, lauric amide ethyldiethylamine oxide, myristic amide ethyldiethylamine oxide, coconut fatty acid amide ethyldiethylamine oxide, lauric amide ethyldihydroxyethylamine oxide, myristic amide ethyldihydroxyethylamine oxide, and coconut fatty acid amide ethyldihydroxyethylamine oxide.

The cosmetic of the present invention can comprise various other raw materials. The aforementioned raw materials are preferably hydrophobic so that they are completely insoluble in water at room temperature or the solubility thereof with respect to 100 g of water is below 1% by weight (mass).

As examples of the aforementioned raw materials, mention may be made of, for example, a silicone resin, a silicone elastomer, a water-soluble polymer, an oil-based gelling agent, an organo-modified clay mineral, a silicone gum, an organo-modified silicone, a UV protective component, and the like.

The silicone resin is an organopolysiloxane with a highly branched molecular structure, a net-like molecular structure or a cage-like molecular structure, and may be in the form of a liquid or solid at room temperature. Any silicone resins usually used in cosmetics can be used unless they are contrary to the purposes of the present invention. In the case of a solid silicone resin, the silicone resin may be in the form of particles such as spherical powders, scale powders, needle powders platy flake powders (including platy powders having an aspect ratio of particles and the outer appearance which are generally understood as a plate form) or the like. In particular, silicone resin powders containing a monoorganosiloxy unit (T unit) and/or a siloxy unit (Q unit) described below are preferably used.

Blending the silicone resin together with the sugar alcohol-modified organopolysiloxane of the present invention is useful, since the miscibility with the oil agents and the uniformly dispersing property can be improved, and at the same time, an effect of improving sensation during use such as uniform adhesiveness with respect to the part to be applied, obtained in accordance with blending the silicone resin can be obtained.

As examples of the solid silicone resins, mention may be made of, for example, MQ resins, MDQ resins, MTQ resins, MDTQ resins, TD resins, TQ resins, or TDQ resins comprising any combinations of a triorganosiloxy unit (M unit) (wherein the organo group is a methyl group alone, or a methyl group in combination with a vinyl group or a phenyl group), a diorganosiloxy unit (D unit) (wherein the organo group is a methyl group alone, or a methyl group in combination with vinyl group or phenyl group), a monoorganosiloxy unit (T unit) (wherein the organo group is a methyl group, a vinyl group or a phenyl group), and a siloxy unit (Q unit). In addition, as other examples thereof, mention may be made of trimethylsiloxysilicic acid, polyalkylsiloxysilicic acid, trimethylsiloxysilicic acid containing dimethylsiloxy units and alkyl(perfluoroalkyl) siloxysilicic acid. The aforementioned silicone resins are preferably oil soluble, and, in particular, preferably are soluble in a volatile silicone.

The silicone resin preferably used in the cosmetic of the present invention possesses at least a monoorganosiloxy unit (T unit) and/or a siloxy unit (Q unit). The aforementioned silicone resin may preferably be blended in an amount ranging from 0.1 to 10% by weight (mass) with respect to the total amount of the cosmetic. The aforementioned silicone resins having branched units have a net-like structure. In the case of applying these to the skin, hair or the like, a uniform film can be formed. Thereby, a protecting effect with respect to dryness and low temperatures can be provided, and make-up running occurred together with sebum by perspiration can be prevented. In addition, the aforementioned silicone resins having the branched units can tightly adhere to the skin, hair or the like, and can provide glossiness and a transparent impression to the skin, hair or the like.

In particular, a phenyl silicone resin with an increased refractive index which has an increased content of a phenyl group (such as 217 Flake Resin manufactured by Dow Corning Toray Co., Ltd.) can easily form silicone resin powders in the form of flakes. In the case of blending the powders in a cosmetic, a brilliant transparent impression can be provided to the skin and hair.

The silicone elastomer can be blended as the aforementioned silicone elastomer powders or a crosslinking organopolysiloxane.

The aforementioned silicone elastomer powders can be used in the cosmetic of the present invention, in the form of an aqueous dispersion. As examples of commercially available products of the aforementioned aqueous dispersions, mention may be made of, for example, “BY 29-129” and “PF-2001 PIF Emulsion” manufactured by Dow Corning Toray Co., Ltd., and the like. By blending an aqueous dispersion (=suspension) of the aforementioned silicone elastomer powders, the sensation during use of the cosmetic, and in particular, oil-in-water emulsion cosmetic can be further improved.

The crosslinking organopolysiloxane preferably has a structure in which an organopolysiloxane chain is three-dimensionally crosslinked by a reaction with a crosslinking component formed from a polyether unit, an alkylene unit having 4 to 20 carbon atoms, and an organopolysiloxane unit, or the like.

The crosslinking organopolysiloxane can be particularly obtained by addition-reacting an organohydrogenpolysiloxane having silicon-binding hydrogen atoms, a polyether compound having unsaturated bonds at both terminals of the molecular chain, an unsaturated hydrocarbon having more than one double bonds in a molecule, and an organopolysiloxane having more than one double bond in a molecule. Here, the crosslinking organopolysiloxane may or may not have a modifying functional group such as an unreacted silicon-binding hydrogen atom, an aromatic hydrocarbon group such as a phenyl group or the like, a long chain alkyl group having 6 to 30 carbon atoms such as an octyl group, a polyether group, a carboxyl group, a silylalkyl group having the aforementioned carbosiloxane dendrimer structure or the like, and can be used without restrictions of physical modes and preparation methods such as dilution, properties and the like.

As one example, the aforementioned crosslinking organopolysiloxane can be obtained by addition-reacting an organohydrogenpolysiloxane which is formed from a structure unit selected from the group consisting of a SiO₂ unit, a HSiO_{1.5} unit, a R^{b}SiO_{1.5} unit, a R^{b}HSiO unit, a R^{b}₂SiO unit, a R^{b}₃SiO_{0.5} unit and a R^{b}₂HSiO_{0.5} unit, wherein R^{b} is a substituted or non-substituted monovalent hydrocarbon group having 1 to 30 carbon atoms, excluding an aliphatic unsaturated group, and a part of R^{b} is a monovalent hydrocarbon group having 8 to 30 carbon atoms, and at the same time, includes 1.5 or more, on average, of hydrogen atoms binding to the silicon atom in the molecule, with a crosslinking component selected from the group consisting of a polyoxyalkylene compound having unsaturated hydrocarbon groups at both terminals of the molecular chain, a polyether compound such as a polyglycerol compound, a polyglycidyl ether compound or the like, an unsaturated hydrocarbon which is an α,ω-diene represented by the following general formula: CH₂═CH—CᵣH₂ᵣ—CH═CH₂, wherein r is an integer ranging from 0 to 26, and an organopolysiloxane which is formed from a SiO₂ unit, a (CH₂═CH)SiO_{1.5} unit, a R^{c}SiO_{1.5} unit, a R^{c}(CH₂═CH)SiO unit, a R^{c}₂SiO unit, a R^{c}₃SiO_{0.5}, and a R^{c}₂(CH₂═CH)SiO_{0.5}, wherein R^{c} is a substituted or non-substituted monovalent hydrocarbon group having 1 to 30 carbon atoms, excluding an aliphatic unsaturated group, and includes 1.5 or more, on average, of vinyl groups binding to the silicon atom in a molecule. The aforementioned modifying functional group can be introduced by carrying out an addition reaction with respect to the unreacted hydrogen atoms binding to the silicon atom. For example, 1-hexene is reacted with a crosslinking organopolysiloxane having an unreacted hydrogen atom binding to the silicon atom, and thereby, a hexyl group which is an alkyl group having 6 carbon atoms can be introduced thereinto.

The aforementioned crosslinking organopolysiloxanes can be used without restrictions of physical modes and preparation methods such as dilution, properties and the like. As particularly preferable examples thereof, mention may be made of α,ω-diene crosslinking silicone elastomers (as commercially available products, DC 9040 Silicone Elastomer Blend, DC 9041 Silicone Elastomer Blend, DC 9045 Silicone Elastomer Blend, and DC 9046 Silicone Elastomer Blend, manufactured by Dow Corning Corporation in the USA) described in U.S. Pat. No. 5,654,362. In the same manner as described above, as examples of partially crosslinking organopolysiloxane polymers, mention may be made of (dimethicone/vinyldimethicone) crosspolymer, (dimethicone/phenylvinyldimethicone) crosspolymer, (PEG-8 to 30/C6 to C30 alkyldimethicone) crosspolymer, (vinyldimethicone/C6 to C30 alkyldimethicone) crosspolymer, (dimethicone/polyglycerol) crosspolymer and the like, in the case of using INCI names (International Nomenclature Cosmetic Ingredient labeling names).

In the case of blending an emulsifiable crosslinking organopolysiloxane formed by crosslinking by means of a polyether compound in a cosmetic as a component, the aforementioned sugar alcohol-modified organopolysiloxane can function as a dispersant. For this reason, there is an advantage in that a uniform emulsification system can be formed.

On the other hand, in the case of blending a non-emulsifiable crosslinking organopolysiloxane formed by crosslinking by means of an unsaturated hydrocarbon group such as a diene or an organopolysiloxane in a cosmetic as a component, an adhesive sensation to the skin can be improved. In addition, there is an advantage in that good compatibility with other oil agents can be exhibited, and the whole oil system can be uniformly and stably blended in the cosmetic.

The aforementioned silicone elastomer can be blended alone or in combination with two or more types thereof in accordance with the purpose thereof. The silicone elastomer may be blended in an amount preferably ranging from 0.05 to 25% by weight (mass) and more preferably ranging from 0.1 to 15% by weight (mass), with respect to the total amount of the cosmetic, in accordance with the purpose and blending intention.

The water-soluble polymer may be blended in order to prepare a cosmetic in the desirable form, and improve sensation during use of the cosmetic such as feeling on touch with respect to hair or the like, a conditioning effect or the like. Any one of amphoteric, cationic, anionic, and nonionic polymers, and water-swellable clay minerals can be used as long as they are commonly used in a cosmetic. One type or two or more types of water-soluble polymers can be used. The aforementioned water-soluble polymers have an effect of thickening a hydrous component, and for this reason, they are useful in the case of obtaining a hydrous cosmetic, and in particular, in the form of a gel, a water-in-oil emulsion cosmetic, and an oil-in-water emulsion cosmetic. As examples of natural water-soluble polymers, mention may be made of vegetable-based polymers such as gum Arabic, tragacanth gum, galactan, guar gum, carob gum, karaya gum, carrageenan, pectin, agar, quince seed, algal colloid, starch (rice, corn, potato, or wheat), glycyrrhizinic acid and the like; microorganism-based polymers such as xanthan gum, dextran, succinoglucan, pullulan, and the like; and animal-based polymers such as collagen, casein, albumin, gelatin, and the like. In addition, as examples of semi-synthetic water-soluble polymers, mention may be made of, for example, starch-based polymers such as carboxymethyl starch, methylhydroxypropyl starch, and the like; cellulose-based polymers such as methylcellulose, nitrocellulose, ethylcellulose, methylhydroxypropylcellulose, hydroxyethylcellulose, sodium cellulose sulfate, hydroxypropylcellulose, sodium carboxymethylcellulose (CMC), crystalline cellulose, cellulose powder, and the like; and alginate-based polymers such as sodium alginate, propylene glycol alginate and the like. As examples of synthetic water-soluble polymers, mention may be made of, for example, vinyl-based polymers such as polyvinyl alcohol, polyvinyl methyl ether-based polymer, polyvinylpyrrolidone, carboxyvinyl polymer (CARBOPOL 940, CARBOPOL 941; manufactured by The Lubrizol Corporation); polyoxyethylene-based polymers such as polyethylene glycol 20,000, polyethylene glycol 6,000, polyethylene glycol 4,000 and the like; copolymer-based polymers such as a copolymer of polyoxyethylene and polyoxypropylene, PEG/PPG methyl ether and the like; acryl-based polymers such as poly(sodium acrylate), poly(ethyl acrylate), polyacrylamide and the like; polyethylene imines; cationic polymers and the like. The water-swellable clay minerals are nonionic water-soluble polymers and correspond to one type of colloid-containing aluminum silicate having a triple layer structure. More particular, as examples thereof, mention may be made of bentonite, montmorillonite, beidellite, nontronite, saponite, hectorite, aluminum magnesium silicate, and silicic anhydride. They may be any one of natural ones and synthesized ones.

As examples of other cationic water-soluble polymers, in particular, as components which are preferably blended in cosmetics for use on hair, mention may be made of quaternary nitrogen-modified polysaccharides such as cation-modified cellulose, cation-modified hydroxyethylcellulose, cation-modified guar gum, cation-modified locust bean gum, cation-modified starch and the like; dimethyldiallylammonium chloride derivatives such as a copolymer of dimethyldiallylammonium chloride and acrylamide, poly(dimethylmethylene piperidinium chloride) and the like; vinylpyrrolidone derivatives such as a salt of a copolymer of vinylpyrrolidone and dimethylaminoethyl methacrylic acid, a copolymer of vinylpyrrolidone and methacrylamide propyltrimethylammonium chloride, a copolymer of vinylpyrrolidone and methylvinylimidazolium chloride and the like; and methacrylic acid derivatives such as a copolymer of methacryloylethyldimethylbetaine, methacryloylethyl trimethylammonium chloride and 2-hydroxyethyl methacrylate, a copolymer of methacryloylethyldimethylbetaine, methacryloylethyl trimethylammonium chloride and methoxy polyethylene glycol methacrylate, and the like.

In addition, in particular, as a component which can be preferably blended in a cosmetic for use on hair, an amphoteric water-soluble polymer can be mentioned. More particularly, as examples thereof, mention may be made of amphoterized starches; dimethyldiallylammonium chloride derivatives such as a copolymer of acrylamide, acrylic acid, and dimethyldiallylammonium chloride, and a copolymer of acrylic acid and dimethyldiallylammonium chloride; and methacrylic acid derivatives such as polymethacryloylethyl dimethylbetaine, a copolymer of methacryloyloxyethyl carboxybetaine and alkyl methacrylate, a copolymer of octylacrylamide, hydroxypropyl acrylate and butylaminoethyl methacrylate, and a copolymer of N-methacryloyloxyethyl-N,N-dimethylammonium α-methylcarboxybetaine and alkyl methacrylate.

The blending amount of the water-soluble polymer in the cosmetic of the present invention can be suitably selected in accordance with the type and purpose of cosmetic. The amount may preferably range from 0.01 to 5.0% by weight (mass), and more preferably may range from 0.1 to 3.0% by weight (mass) with respect to the total amount of the cosmetic in order to particularly obtain a superior sensation during use. If the blending amount of the water-soluble polymer exceeds the aforementioned upper limit, a rough feeling with respect to the hair or skin may remain in some types of the cosmetics. On the other hand, if the blending amount is below the aforementioned lower limit, advantageous technical effects such as a thickening effect, a conditioning effect and the like may not be sufficiently exhibited.

The oil-soluble gelling agent is a gelling agent of an oil agent. In the case in which the cosmetic of the present invention is an oil-based cosmetic having an oil agent as a continuous phase, by thickening/gelling the oil-based component, the desirable formulation and feeling on touch can be obtained. As examples of oil-soluble gelling agents, mention may be made of metallic soaps such as aluminum stearate, magnesium stearate, zinc myristate and the like; amino acid derivatives such as N-lauroyl-L-glutamic acid, α,γ-di-n-butylamine and the like; dextrin fatty acid esters such as dextrin palmitate, dextrin stearate, dextrin 2-ethylhexanoate palmitate and the like; sucrose fatty acid esters such as sucrose palmitate, sucrose stearate and the like; benzylidene derivatives of sorbitol such as monobenzylidene sorbitol, dibenzylidene sorbitol and the like; and the like. The oil-soluble gelling agents can be used alone or in combination of two or more types thereof, if necessary.

The usage amount of the oil-soluble gelling agent in the cosmetic of the present invention is not particularly restricted, and may preferably range from 0.5 to 50 parts by weight (mass) and more preferably may range from 1 to 30 parts by weight (mass), with respect to 100 parts by weight (mass) of the oil agent(s).

When the oil-soluble gelling agent is used in the cosmetic in which the aforementioned sugar alcohol-modified organopolysiloxane, powder(s) and oil agent(s) are blended, there are advantages in view of qualities in that an oily sensation (oily and sticky feeling on touch) can be further totally controlled, and cosmetic durability can be further improved.

The organo-modified clay mineral can be used as a gelling agent for the oil agent(s) in the same manner as described in the aforementioned oil-soluble gelling agent. As examples of organo-modified clay minerals, mention may be made of, for example, dimethylbenzyl dodecylammonium montmorillonite clay, dimethyldioctadecylammonium montmorillonite clay, dimethylalkylammonium hectorite, benzyldimethylstearylammonium hectorite, distearyldimethylammonium chloride-treated aluminum magnesium silicate and the like. As examples of commercially available products thereof, mention may be made of Benton 27 (benzyldimethylstearylammonium chloride-treated hectorite, manufactured by Nationalred Co.), Benton 38 (distearyldimethylammonium chloride-treated hectorite, manufactured by Nationalred Co.) and the like.

The silicone gum is a linear diorganopolysiloxane having an ultra-high degree of polymerization, and is also referred to as a silicone raw rubber or an organopolysiloxane gum. The silicone raw rubber possesses a high degree of polymerization, and for this reason, it has a measurable degree of plasticity. In view of this, the silicone gum is different from the aforementioned oil silicones. The aforementioned silicone gum can be blended in the cosmetic according to the present invention as it is, or as a liquid gum dispersion (an oil dispersion of the silicone gum) in which the silicone gum is dispersed in an oil silicone.

As examples of the aforementioned silicone raw rubber, mention may be made of substituted or non-substituted organopolysiloxanes having a dialkylsiloxy unit (D unit) such as dimethylpolysiloxane, methylphenylpolysiloxane, aminopolysiloxane, methylfluoroalkyl polysiloxane and the like, or those having a micro-crosslinking structure thereof and the like. As representative examples thereof, there are those represented by the following general formula:

R⁷(CH₃)₂SiO{(CH₃)₂SiO}ₛ{(CH₃)R⁸SiO}ₜSi(CH₃)₂R⁷

wherein R⁸ is a group selected from a vinyl group, a phenyl group, an alkyl group having 6 to 20 carbon atoms, an aminoalkyl group having 3 to 15 carbon atoms, a perfluoroalkyl group having 3 to 15 carbon atoms, and a quaternary ammonium salt group-containing alkyl group having 3 to 15 carbon atoms; the terminal group R⁷ is a group selected from an alkyl group having 1 to 8 carbon atoms, a phenyl group, a vinyl group, an aminoalkyl group having 3 to 15 carbon atoms, a hydroxyl group and an alkoxy group having 1 to 8 carbon atoms; s=2,000 to 6,000; t=0 to 1,000; and s+t=2,000 to 6,000. Among these, a dimethylpolysiloxane raw rubber having a degree of polymerization ranging from 3,000 to 20,000 is preferred. In addition, an amino-modified methylpolysiloxane raw rubber having a 3-aminopropyl group, an N-(2-aminoethyl)-3-aminopropyl group or the like on the side chain or the terminal of the molecule is preferred. In addition, in the present invention, the silicone gum can be used alone or in combination with two or more types thereof, as necessary.

The silicone gum has an ultra-high degree of polymerization. For this reason, the silicone gum can exhibit a superior retention property on hair or skin, and can form a protective film with a superior aeration property. For this reason, the silicone gum is a component which can particularly provide glossiness and luster on hair and can impart a texture with tension on the entire hair during use and after use.

The blending amount of the silicone gum may range from 0.05 to 30% by weight (mass) and may preferably range from 1 to 15% by weight (mass), with respect to the total amount of the cosmetic. When the silicone gum is used as an emulsion composition prepared via a step of preliminarily emulsifying (including emulsion polymerization), the silicone gum can be easily blended, and can stably be blended in the cosmetic of the present invention. If the blending amount of the silicone gum is below the aforementioned lower limit, an effect of imparting specific feeling on touch or glossiness with respect to hair may be insufficient.

The organo-modified silicone is a silicone compound in which a functional group is introduced into a part of the polysiloxane chain, is an organo-modified silicone other than the aforementioned sugar alcohol-modified organopolysiloxane, and can be blended in the cosmetic. As examples thereof, mention may be made of an amino-modified silicone, an aminopolyether-modified silicone, an epoxy-modified silicone, a carboxy-modified silicone, an amino acid-modified silicone, acryl-modified silicone, a phenol-modified silicone, an amidoalkyl-modified silicone, an aminoglycol-modified silicone, an alkoxy-modified silicone, and a silicone modified with a higher alkyl group having 8 to 30 carbon atoms.

The organo-modified silicone may have an alkylene chain, an aminoalkylene chain, or a polyether chain, in addition to a polysiloxane bond as a main chain, and includes so-called block copolymer. In addition, the aforementioned organo-modified group(s) may be present at the side chain(s) or at one or both of the terminal(s) of the polysiloxane chain.

The organo-modified silicone can be blended alone or in combination with two or more types thereof in accordance with the purpose thereof. A function as the aforementioned silicone-based surfactant, a function as a powder treatment agent, an effect of improving smoothness and glossiness with respect to hair, and in particular, a function of improving feeling on touch after rinsing the hair, and the like can be exhibited. In the cosmetic of the present invention, the blending amount of the organo-modified silicone is not particularly restricted, and may preferably range from 0.05 to 25% by weight (mass) and more preferably may range from 0.1 to 15% by weight (mass), with respect to the total amount of the cosmetic, in accordance with the purpose and blending intention thereof. If the amount is below the aforementioned lower limit, the desirable function of the organo-modified silicone may not be sufficiently exhibited. On the other hand, if the amount exceeds the aforementioned upper limit, a balance of feeling on touch, functions, and the like of the cosmetic may be impaired.

A UV-ray protective component is a component for blocking or diffusing UV rays. Among UV-ray protective components, there are inorganic UV-ray protective components and organic UV-ray protective components. If the cosmetics of the present invention are sunscreen cosmetics, at least one type of inorganic or organic UV-ray protective component, and in particular, an organic UV-ray protective component is preferably contained.

The inorganic UV-ray protective components may be components in which the aforementioned inorganic powder pigments, metal powder pigments and the like are blended as UV-ray dispersants. As examples thereof, mention may be made of metal oxides such as titanium oxide, zinc oxide, cerium oxide, titanium suboxide, iron-doped titanium oxides and the like; metal hydroxides such as iron hydroxides and the like; metal flakes such as platy iron oxide, aluminum flake and the like; and ceramics such as silicon carbide and the like. Among these, at least one type of a material selected from fine particulate metal oxides and fine particulate metal hydroxides with an average particle size ranging from 1 to 100 nm in the form of granules, plates, needles, or fibers is, in particular, preferred. The aforementioned powders are preferably subjected to conventional surface treatments such as fluorine compound treatments, among which a perfluoroalkyl phosphate treatment, a perfluoroalkylsilane treatment, a perfluoropolyether treatment, a fluorosilicone treatment, and a fluorinated silicone resin treatment are preferred; silicone treatments, among which a methylhydrogenpolysiloxane treatment, a dimethylpolysiloxane treatment, and a vapor-phase tetramethyltetrahydrogencyclotetrasiloxane treatment are preferred; silicone resin treatments, among which a trimethylsiloxysilicic acid treatment is preferred; pendant treatments which are methods of adding alkyl chains after the vapor-phase silicone treatment; silane coupling agent treatments; titanium coupling agent treatments; silane treatments among which an alkylsilane treatment and an alkylsilazane treatment are preferred; oil agent treatments; N-acylated lysine treatments; polyacrylic acid treatments; metallic soap treatments in which a stearic acid salt or a myristic acid salt is preferably used; acrylic resin treatments; metal oxide treatments and the like. Multiple treatments described above are preferably carried out. For example, the surface of the fine particulate titanium oxide can be coated with a metal oxide such as silicon oxide, alumina or the like, and then, a surface treatment with an alkylsilane can be carried out. The total amount of the material used for the surface treatment may preferably range from 0.1 to 50% by weight (mass) based on the amount of the powder.

The organic UV-ray protective components are generally lipophilic. More particularly, as examples of the aforementioned organic UV-ray protective components, mention may be made of benzoic acid-based UV-ray absorbers such as paraminobenzoic acid (hereinafter, referred to as PABA), PABA monoglycerol ester, N,N-dipropoxy-PABA ethyl ester, N,N-diethoxy-PABA ethyl ester, N,N-dimethyl-PABA ethyl ester, N,N-dimethyl-PABA butyl ester, 2-[4-(diethylamino)-2-hydroxybenzoyl]benzoic acid hexyl ester (trade name: Uvinul A Plus) and the like; anthranilic acid-based UV-ray absorbers such as homomethyl N-acetylanthranilate and the like; salicylic acid-based UV-ray absorbers such as amyl salicylate, menthyl salicylate, homomethyl salicylate, octyl salicylate, phenyl salicylate, benzyl salicylate, p-isopropanolphenyl salicylate and the like; cinnamic acid-based UV-ray absorbers such as octyl cinnamate, ethyl 4-isopropylcinnamate, methyl 2,5-diisopropylcinnamate, ethyl 2,4-diisopropylcinnamate, methyl 2,4-diisopropylcinnamate, propyl p-methoxycinnamate, isopropyl p-methoxycinnamate, isoamyl p-methoxycinnamate, octyl p-methoxycinnamate (2-ethylhexyl p-methoxycinnamate), 2-ethoxyethyl p-methoxycinnamate, cyclohexyl p-methoxy cinnamate, ethyl α-cyano-β-phenylcinnamate, 2-ethylhexyl α-cyano-β-phenylcinnamate, glyceryl mono-2-ethylhexanoyl-diparamethoxycinnamate, 3-methyl-4-[methylbis(trimethylsiloxy)silyl]butyl 3,4,5-trimethoxycinnamate, dimethicodiethyl benzal malonate (trade name: Parsol SLX (INCI name=polysilicone-15) and the like; benzophenone-based UV-ray absorbers such as 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone 5-sulfonate, 4-phenylbenzophenone, 2-ethylhexyl-4′-phenylbenzophenone 2-carboxylate, hydroxy-4-n-octoxybenzophenone, 4-hydroxy-3-carboxybenzophenone and the like; 3-(4′-methylbenzylidene)-d,l-camphor; 3-benzylidene-d,l-camphor; urocanic acid; ethyl urocanate; 2-phenyl-5-methylbenzoxazole; benzotriazole-based UV-ray absorbers such as 2,2′-hydroxy-5-methylphenyl benzotriazole, 2-(2′-hydroxy-5′-t-octylphenyl)benzotriazole; 2-(2′-hydroxy-5′-methylphenyl)benzotriazole; dibenzaladine; dianisoylmethane; 4-methoxy-4′-t-butylbenzoylmethane, 5-(3,3-dimethyl-2-norbonylidene)-3-pentan-2-one, 2,2′-methylenebis(6-(2H-benzotriazol-2-yl)-4-(1,1,3,3-tetramethylbutyl)phenol) (trade name: trademark TINOSORB M) and the like; triazine-based UV-ray absorbers such as 2,4,6-tris[4-(2-ethylhexyloxycarbonyl)anilino]1,3,5-triazine (INCI: octyltriazone), 2,4-bis{[4-(2-ethyl-hexyloxy)-2-hydroxy]phenyl}-6-(4-methoxyphenyl)-1,3,5-triazine (INCI: bis-ethylhexyloxyphenol methoxyphenyl triazine, trade name: trademark TINOSORB S) and the like; 2-ethylhexyl 2-cyano-3,3-diphenylprop-2-enoate (INCI: octocrylene) and the like.

Furthermore, hydrophobic polymer powders containing the aforementioned organic UV-ray protective components inside thereof can also be used. The polymer powder may be hollow or not, may have an average primary particle size thereof ranging from 0.1 to 50 μm and may have a particle size distribution thereof of either broad or sharp. As examples of the polymers, mention may be made of acrylic resins, methacrylic resins, styrene resins, polyurethane resins, polyethylene, polypropylene, polyethylene terephthalate, silicone resins, nylons, acrylamide resins, and silylated polypeptide resins. Polymer powders containing the organic UV-ray protective components in the amount ranging from 0.1 to 30% by weight (mass) with respect to the amount of the powder are preferred. Polymer powders containing 4-tert-butyl-4′-methoxydibenzoylmethane, which is a UV-A absorber, are particularly preferred.

The UV-ray protective components which can be preferably used in the cosmetics of the present invention may be at least one type of compound selected from the group consisting of fine particulate titanium oxide, fine particulate zinc oxide, 2-ethylhexyl paramethoxycinnamate, 4-tert-butyl-4′-methoxydibenzoylmethane, benzotriazole-based UV-ray absorbers and triazine-based UV-ray absorbers. The aforementioned UV-ray protective components are commonly used and easily available, and exhibit superior effects of preventing ultraviolet rays. For these reasons, the aforementioned UV-ray protective components are preferably used. In particular, inorganic UV-ray protective components and organic UV-ray protective components are preferably used in combination. In addition, UV-A protective components and UV-B protective components are further preferably used in combination.

In the cosmetic of the present invention, by use of the aforementioned sugar alcohol-modified organopolysiloxane together with the UV-ray protective component(s), the whole feeling on touch and storage stability of the cosmetic can be improved, and at the same time, the UV-ray protective component(s) can stably dispersed in the cosmetic. For this reason, superior UV-ray protective functions can be provided to the cosmetic.

In the cosmetic of the present invention, the aforementioned UV-ray protective component(s) may be blended in a total amount preferably ranging from 0.1 to 40.0% by weight (mass), and more preferably ranging from 0.5 to 15.0% by weight (mass), with respect to the total amount of the cosmetic.

In addition, in the cosmetic of the present invention, at least one material selected from the group consisting of acryl silicone dendrimer copolymers, polyamide-modified silicones, alkyl-modified silicone waxes, and alkyl-modified silicone resin waxes may be blended.

Acryl silicone dendrimer copolymers are vinyl-based polymer having a carbosiloxane dendrimer structure at the side chain. As examples thereof, mention may be, in particular, preferably made of vinyl-based polymers described in Japanese Patent No. 4,009,382 (Japanese Unexamined Patent Application, First Publication No. 2000-063225). As examples of commercially available products, mention may be made of FA 4001 CM Silicone Acrylate, and FA 4002 ID Silicone Acrylate, manufactured by Dow Corning Toray Co., Ltd., and the like. An acryl silicone dendrimer copolymer having a long chain alkyl group having 8 to 30 carbon atoms and preferably having 14 to 22 carbon atoms at the side chain or the like may be used. In the case of blending the aforementioned acryl silicone dendrimer copolymer alone, a superior property of forming a film can be exhibited. For this reason, by blending the dendrimer copolymer in the cosmetic according to the present invention, a strong coating film can be formed on the applied part, and durability of a sebum resistance property, a rub resistance property and the like can be considerably improved.

By using the aforementioned sugar alcohol-modified organopolysiloxane together with an acryl silicone dendrimer copolymer, there are advantages in that a surface protective property such as a sebum resistance property can be improved due to a strong water repellency provided by the carbosiloxane dendrimer structure; and at the same time, irregularities such as pores and wrinkles of the skin to be applied can be effectively made inconspicuous. In addition, the aforementioned sugar alcohol-modified organopolysiloxane can exhibit a superior dispersion property with respect to the other oil agent(s) and powder(s), and can provide miscibility of an acryl silicone dendrimer copolymer with the other oil agent(s). For this reason, there is an advantage in that makeup running or gathering on the skin can be controlled for a long time. In addition, when powders are treated in accordance with a conventional method by using the aforementioned sugar alcohol-modified organopolysiloxane together with the acryl silicone dendrimer copolymer, a raw material for a cosmetic (powder composition for use in a cosmetic) exhibiting superior blending stability can be prepared.

The blending amount of the acryl silicone dendrimer copolymer can appropriately be selected in accordance with the purpose and blending intention. The amount may preferably range from 1 to 99% by weight (mass), and more preferably may range from 30 to 70% by weight (mass), with respect to the total amount of the cosmetic.

As examples of polyamide-modified silicones, mention may be made of, for example, siloxane-based polyamides described in U.S. Pat. No. 5,981,680 (Japanese Unexamined Patent Application, First Publication No. 2000-038450) and Published Japanese Translation No. 2001-512164 of the PCT International Application, and as examples of commercially available products, mention may be made of 2-8178 Gellant, 2-8179 Gellant and the like (manufactured by Dow Corning Corporation, in the USA). The aforementioned polyamide-modified silicones are useful as an oil-based raw material, and in particular, a thickening/gelling agent of a silicone oil in the same manner as described in the aforementioned oil-soluble gelling agent.

In the case of using the polyamide-modified silicone together with the aforementioned sugar alcohol-modified organopolysiloxane, compatibility with the oil agent such as a silicone oil or the like can be further improved. For this reason, the cosmetic according to the present invention can exhibit a good spreading property, a good styling property, a superior stable sensation and a superior adhesive property in the case of applying to the skin, hair or the like. In addition, there are advantages in view of qualities in that a glossy transparent sensation and superior gloss can be provided, the viscosity or hardness (flexibility) of the whole cosmetic containing oil-based raw material(s) can be appropriately adjusted, and an oily sensation (oily and sticky feeling on touch) can be totally controlled. In addition, by use of the aforementioned sugar alcohol-modified organopolysiloxane, dispersion stability of perfume(s), powder(s) and the like can be improved. For this reason, for example, there is a characteristic in that a uniform and fine cosmetic sensation can be maintained for a long time.

The usage amount of the polyamide-modified silicone may appropriately be selected in accordance with the purpose and blending intention. In the case of using as a gelling agent of an oil-based raw material, the amount may preferably range from 0.5 to 80 parts by weight (mass) and more preferably may range from 1 to 50 parts by weight (mass), with respect to 100 parts by weight (mass) of the oil agent.

The alkyl-modified silicone waxes are components useful as a part of a base material of an oil-based solid cosmetic. In the cosmetic of the present invention, an alkyl-modified silicone in the form of a wax at room temperature can be used without particular restrictions. As examples thereof, mention may be made of a methyl(long chain alkyl)polysiloxane having both molecular terminals capped with trimethylsiloxy groups, a copolymer of a dimethylpolysiloxane having both molecular terminals capped with trimethylsiloxy groups and a methyl(long chain alkyl)siloxane, a dimethylpolysiloxane modified with long chain alkyls at both terminals, and the like. As examples of commercially available products thereof, mention may be made of, AMS-C30 Cosmetic Wax, 2503 Cosmetic Wax and the like (manufactured by Dow Corning Corporation, in the USA).

The aforementioned sugar alcohol-modified organopolysiloxane can exhibit a superior dispersion property of an alkyl-modified silicone wax, and for this reason, a cosmetic exhibiting superior storage stability for a long time can be obtained. In addition, a superior forming property of the cosmetic can also be exhibited. In particular, in a system containing powder(s), there is an advantage in that separation of the alkyl-modified silicone wax hardly occurs, and an oil-based cosmetic which can exhibit superior form-retaining strength and can be smoothly and uniformly spread during application can be provided.

In the present invention, the alkyl-modified silicone wax preferably has a melting point of 60° C. or higher in view of a makeup durability effect and stability at increased temperatures. The blending amount thereof can appropriately be selected in accordance with the purpose and blending intention, and usually ranges from 1 to 50% by weight (mass) with respect to the total amount of the cosmetic. In order to improve formability and cosmetic durability of the oil-based cosmetic, the alkyl-modified silicone wax may be blended in an amount more preferably ranging from 5 to 40% by weight (mass). In addition, the alkyl-modified silicone wax can exhibit rich compatibility with the silicone oil(s) having a long-chain alkyl group such as the aforementioned alkyl-modified silicone or the like, and crosslinking organopolysiloxanes. For this reason, the aforementioned optional components are preferably used.

The alkyl-modified silicone resin wax is a component for imparting sebum durability, a moisture-retaining property, and a fine texture feeling on touch to the cosmetic. For example, a silsesquioxane resin wax described in Published Japanese Translation No. 2007-532754 of the PCT International Application may be mentioned. As commercially available products thereof, SW-8005 C30 RESIN WAX (manufactured by Dow Corning Corporation in the USA) and the like may be mentioned.

The aforementioned sugar alcohol-modified organopolysiloxane can uniformly disperse the alkyl-modified silicone resin wax in the cosmetic, in the same manner as described in the alkyl-modified silicone wax. In addition, an oil phase containing the aforementioned alkyl-modified silicone resin wax can be stably emulsified optionally together with the other surfactant. A conditioning effect with respect to skin or hair can be improved and a fine texture and a moisturized feeling on touch can be imparted.

In the cosmetic of the present invention, the blending amount of the alkyl-modified silicone resin wax may appropriately be selected in accordance with the purpose and blending intention. The amount can usually range from 0.5 to 50% by weight (mass) with respect to the total amount of the cosmetic. In order to realize sebum durability and a fine texture feeling on touch of the cosmetic, the blending amount may preferably range from 1 to 30% by weight (mass).

In the cosmetics of the present invention, other components usually used in cosmetics can be blended within a range which does not impair the effects of the present invention, such as alcohols, organic resins, moisture-retaining agents, thickening agents, preservatives, anti-microbial agents, perfumes, salts, antioxidants, pH adjusting agents, chelating agents, algefacients, anti-inflammatory agents, physiologically active components, components for beautifying the skin (such as whitening agents, cell activators, agents for ameliorating skin roughness, blood circulation accelerators, astringents, antiseborrheic agents and the like), vitamins, amino acids, nucleic acids, hormones, clathrate compounds and the like. They are not particularly restricted thereto.

As the alcohols, one type or two or more types of polyhydric alcohols and/or lower monovalent alcohols can be used. As examples of lower alcohols, mention may be made of ethanol, isopropanol, n-propanol, t-butanol, s-butanol and the like. As examples of polyhydric alcohols, mention may be made of divalent alcohols such as 1,3-propanediol, 1,3-butylene glycol, 1,2-butylene glycol, propylene glycol, trimethylene glycol, tetramethylene glycol, 2,3-butylene glycol, pentamethylene glycol, 2-butene-1,4-diol, dibutylene glycol, pentyl glycol, hexylene glycol, octylene glycol and the like; trivalent alcohols such as glycerol, trimethylolpropane, 1,2,6-hexanetriol and the like; polyhydric alcohols having tetra- or more valences such as pentaerythritol, xylitol and the like; sugar alcohols such as sorbitol, mannitol, maltitol, maltotriose, sucrose, erytritol, glucose, fructose, starch-decomposed products, maltose, xylitose, starch-decomposed reduction alcohols and the like. In addition to the aforementioned polyhydric alcohols having a low molecular weight, mention may be made of polyhydric alcohol polymers such as diethylene glycol, dipropylene glycol, triethylene glycol, polypropylene glycol, tetraethylene glycol, diglycerol, polyethylene glycol, triglycerol, tetraglycerol, polyglycerol and the like. Among these, 1,3-propanediol, 1,3-butylene glycol, sorbitol, dipropylene glycol, glycerol, and polyethylene glycol are, in particular, preferred. The blending amount thereof may preferably range from 0.1 to 50% by weight (mass) with respect to the total amount of the cosmetic. The alcohol(s) can be blended in order to improve storage stability of the cosmetic, in an amount ranging from about 5 to 30% by weight (mass), with respect to the total amount of the cosmetic. This is one of the preferable modes for carrying out the present invention.

As examples of organic resins, mention may be made of polyvinyl alcohol, polyvinyl pyrrolidone, poly(alkyl acrylate) copolymers, and the like. The organic resin possesses a superior property of forming a film. For this reason, by blending the organic resin in the cosmetic of the present invention, a strong coating film can be formed at the applied part, and durability such as sebum resistance and rub resistance or the like can be improved.

As examples of humectants, mention may be made of, for example, hyaluronic acid, chondroitin sulfate, pyrrolidone carboxylic acid salts, polyoxyethylene methylglucoside, polyoxypropylene methylglucoside, and the like. Needless to say, the aforementioned polyhydric alcohols exhibit a function of retaining moisture on the skin or hair.

As examples of the preservatives, mention may be made of, for example, alkyl paraoxybenzoates, benzoic acid, sodium benzoate, sorbic acid, potassium sorbate, phenoxyethanol and the like. As examples of the antimicrobial agents, mention may be made of benzoic acid, salicylic acid, carbolic acid, sorbic acid, alkyl paraoxybenzoates, parachloromethacresol, hexachlorophene, benzalkonium chloride, chlorhexidine chloride, trichlorocarbanilide, trichlosan, photosensitizers, isothiazolinone compounds such as 2-methyl-4-isothiazolin-3-one, 5-chloro-2-methyl-4-isothiazolin-3-one and the like, amine oxides such as dimethyl laurylamine oxide, dihydroxyethyl laurylamine oxide and the like, and the like.

In addition, as examples of anti-microbial agents, mention may be made of apolactoferrin; phenol-based compounds such as resorcinol; anti-microbial or fungicidal basic proteins or peptides such as iturin-based peptides, surfactin-based peptides, protamine or salts thereof (protamine sulfate and the like) and the like; polylysines such as ε-polylysine or salts thereof, and the like; anti-microbial metal compounds which can produce a silver ion, a copper ion or the like; antimicrobial enzymes such as protease, lipase, oxydoreductase, carbohydrase, transferase, phytase and the like; and the like.

As examples of perfume, mention may be made of perfume extracted from flowers, seeds, leaves, and roots of various plants; perfume extracted from seaweeds; perfume extracted from various parts or secretion glands of animals such as musk and sperm oil; or artificially synthesized perfume such as menthol, musk, acetate, and vanilla. The conventional perfume can be selected and blended in an appropriate amount in accordance with the formulations of the cosmetics in order to provide a certain aroma or scent to the cosmetics, or in order to mask unpleasant odor.

As examples of antioxidants, mention may be made of, for example, tocopherol, butylhydroxyanisole, dibutylhydroxytoluene, phytic acid and the like.

As examples of pH adjustors, mention may be made of, for example, lactic acid, citric acid, glycolic acid, succinic acid, tartaric acid, dl-malic acid, potassium carbonate, sodium hydrogencarbonate, ammonium hydrogencarbonate and the like.

As examples of chelating agents, mention may be made of, for example, alanine, sodium salt of edetic acid, sodium polyphosphate, sodium metaphosphate, phosphoric acid and the like.

As examples of algefacients, mention may be made of l-menthol, camphor and the like.

As examples of physiologically active components, mention may be made of, for example, vitamins, amino acids, nucleic acids, hormones, components extracted from natural vegetables, seaweed extracted components, herbal medicine components, whitening agents such as placenta extracts, arbutin, glutathione, saxifrageous extracts and the like; cell activators such as royal jelly, and the like; agents for ameliorating skin roughness; blood circulation accelerators such as nonylic acid vanillylamide, benzyl nicotinate, beta-butoxyethyl nicotinate, capsaicin, gingerone, cantharide tincture, ichthammol, caffeine, tannic acid, alpha-borneol, tocopherol nicotinate, inositol hexanicotinate, cyclandelate, cinnarizine, tolazoline, acetylcholine, verapamil, cepharanthine, gamma-orizanol and the like; astringents such as zinc oxide, tannic acid and the like; antiseborrheic agents such as sulfur, thianthol and the like; anti-inflammatory agents such as ε-aminocaproic acid, glycyrrhizinic acid, β-glycyrrhetinic acid, lysozyme chloride, guaiazulene, hydrocortisone, allantoin, tranexamic acid, azulene and the like; and the like.

As examples of vitamins, mention may be made of vitamin As such as vitamin A oil, retinol, retinol acetate, retinol palmitate and the like; vitamin Bs such as vitamin B2s such as riboflavin, riboflavin butyrate, flavin adenine dinucleotide and the like; vitamin B6s such as pyridoxine hydrochloride, pyridoxine dioctanoate, pyridoxine tripalmitate and the like; vitamin B12 and derivatives thereof; vitamin B15 and derivatives thereof, and the like; vitamin Cs such as L-ascorbic acid, L-ascorbyl dipalmitic acid esters, sodium L-ascorbyl 2-sulfate, dipotassium L-ascorbyl phosphoric acid diester and the like; vitamin Ds such as ergocalciferol, cholecalciferol and the like; vitamin Es such as alpha-tocopherol, beta-tocopherol, gamma-tocopherol, dl-alpha-tocopherol acetate, dl-alpha-tocopherol nicotinate, dl-alpha-tocopherol succinate and the like; vitamin H; vitamin P; nicotinic acids such as nicotinic acid, benzyl nicotinate and the like; pantothenic acids such as calcium pantothenate, D-pantothenyl alcohol, pantothenyl ethyl ether, acetyl pantothenyl ethyl ether and the like; and the like.

As examples of amino acids, mention may be made of glycine, valine, leucine, isoleucine, serine, threonine, phenylalanine, arginine, lysine, aspartic acid, glutamate, cystine, cysteine, methionine, tryptophan and the like.

As examples of nucleic acids, mention may be made of deoxyribonucleic acid and the like.

As examples of hormones, mention may be made of estradiol, ethenyl estradiol and the like.

In the cosmetic of the present invention, natural vegetable extract components, seaweed extract components and herbal medicine components can be blended in accordance with the purposes thereof. As the aforementioned components, in particular, one or more types of components having effects such as whitening effects, anti-ageing effects, effects of ameliorating ageing, effects of beautifying skin, anti-microbial effects, preservative effects and the like can be preferably blended.

As detailed examples thereof, mention may be made of, for example, Angelica keiskei extract, avocado extract, Hydrangea serrata extract, Althaea officinalis extract, Arnica montana extract, aloe extract, apricot extract, apricot kernel extract, Gingko biloba extract, fennel fruit extract, turmeric root extract, oolong tea extract, Rosa multiflora extract, Echinacea angustifolia leaf extract, Scutellaria baicalensis root extract, Phellodendron amurense bark extract, Coptis rhizome extract, Hordeum vulgare seed extract, Hypericum perforatum extract, Lamium album extract, Nasturtium officinale extract, orange extract, dried sea water solution, seaweed extract, hydrolyzed elastin, hydrolyzed wheat powders, hydrolyzed silk, Chamomilla recutita extract, carrot extract, Artemisia capillaris flower extract, Glycyrrhiza glabra extract, Hibiscus sabdariffa extract, Pyracantha fortuneana extract, kiwi extract, Cinchona succirubra extract, cucumber extract, guanosine, Gardenia florida extract, Sasa veitchii extract, Sophora angustifolia extract, walnut extract, grapefruit extract, Clematis vitalba leaf extract, chlorella extract, Morus alba extract, Gentiana lutea extract, black tea extract, yeast extract, burdock extract, fermented rice bran extract, rice germ oil, Symphytum officinale leaf extract, collagen, Vaccinum vitis idaea extract, Asiasarum sieboldi extract, Bupleurum falcatum extract, umbilical extract, Salvia extract, Crocus sativus flower extract, sasa bamboo grass extract, Crataegus cuneata fruit extract, Zanthoxylum piperitum extract, Corthellus shiitake extract, Rehmannia chinensis root extract, Lithospermum erythrorhizone root extract, Perilla ocymoides extract, Tilia cordata extract, Spiraea ulmaria extract, Paeonia albiflora extract, Acorns calamus root extract, Betula alba extract, Equisetum arvense extract, Hedera helix extract, Crataegus oxyacantha extract, Sambucus nigra extract, Achillea millefolium extract, Mentha piperita leaf extract, sage extract, Malva sylvestris extract, Cnidium officinale root extract, Swertia japonica extract, soybean seed extract, Zizyphus jujuba fruit extract, thyme extract, Camellia sinensis leaf extract, Eugenia caryophyllus flower extract, Imperata cylindrica extract, Citrus unshiu peel extract, Angelica acutiloba root extract, Calendula officinalis extract, Prunus persica kernel extract, Citrus aurantium peel extract, Houttuynia cordata extract, tomato extract, natto extract, carrot extract, garlic extract, Rosa canina fruit extract, hibiscus extract, Ophiopogon japonicus root extract, Nelumbo nucifera extract, parsley extract, honey, Hamamelis virginiana extract, Parietaria officinalis extract, Isodon richocarpus extract, bisabolol, Eriobotrya japonica extract, Tussilago farfara flower extract, Petasites japonicus extract, Poria cocos extract, Ruscus aculeatus root extract, grape extract, propolis, Luffa cylindrica fruit extract, safflower flower extract, peppermint extract, Tillia miquellana extract, Paeonia suffruticosa root extract, Humulus lupulus extract, Pinus sylvestris cone extract, horse chestnut extract, Lysichiton camtschatcense extract, Sapindus mukurossi peel extract, Melissa officinalis leaf extract, peach extract, Centaurea cyanus flower extract, Eucalyptus globulus leaf extract, Saxifraga sarementosa extract, Citrus junos extract, Coix lacryma-jobi seed extract, Artemisia princeps extract, lavender extract, apple extract, lettuce extract, lemon extract, Astragalus sinicus extract, rose extract, rosemary extract, Roman chamomile extract, and royal jelly extract. The aforementioned extracts may be water-soluble or oil-soluble.

In the cosmetics of the present invention, depending on the purposes thereof, solvents such as light isoparaffins, ethers, LPG, N-methylpyrrolidone, next-generation chlorofluorocarbons, and the like, can be blended in addition to water such as purified water, mineral water and the like.

The forms of the cosmetics of the present invention are not particularly restricted and can be liquids, milky lotions, creams, solids, pastes, gels, powders, lamellas, mousses, sprays, sheets, and the like. As examples of the cosmetics of the present invention, mention may be made of, for example, UV-ray protective products such as sunscreen agents and the like; skin care products such as cosmetic lotions, cosmetic milks, creams, cleansing products, products for use in massaging, cleansing agents and the like; makeup products such as foundations, makeup bases, cheek colors, eye shadows, mascaras, eyeliners, lipsticks and the like; products for use on hair such as shampoos, rinses, treatments and the like; antiperspirant products; deodorant products and the like.

### EXAMPLES

Hereinafter, the present invention will be described in detail with reference to examples. It should be understood that the present invention is not restricted to the examples. An Me₃SiO group (or an Me₃Si group) is indicated as “M”, an Me₂SiO group is indicated as “D”, an Me₂HSiO group is indicated as “M^{H}”, an MeHSiO group is indicated as “D^{H}”, and units in which a methyl group (Me) in M and D is modified by any substituent are respectively indicated as “M^{R}” and “D^{R}”.

In addition, the xylitol monoallyl ether and the xylitol residue described in the following Reference Examples are the same raw material and functional group as described in the specification of the present application. More particularly, the xylitol monoallyl ether is a raw material comprising xylitol monoallyl ethers represented by the following structural formula: CH₂═CH—CH₂—OCH₂[CH(OH)]₃CH₂OH and represented by the following structural formula: CH₂═CH—CH₂—OCH{CH(OH)CH₂OH}₂ in a weight (mass) ratio of 9:1. In the sugar alcohol-modified silicone of the present invention, the xylitol residue of —C₃H₆—OCH₂[CH(OH)]₃CH₂OH or —C₃H₆—OCH{CH(OH)CH₂OH}₂ corresponding thereto is introduced in the same weight (mass) ratio as described above.

### Reference Example 1

### Synthesis of Sugar Alcohol-Modified Silicone 1

224.6 g of a methylhydrogenpolysiloxane represented by the following average compositional formula: MD₇₀D^{H}₃M, 30.5 g of xylitol monoallyl ether, and 75.0 g of isopropyl alcohol (IPA) were placed in a reactor, and the mixture was heated to 80° C. under a nitrogen stream while it was stirred. 30 mg of a platinum catalyst was added thereto, and the mixture was reacted for 2.5 hours at 80° C. It was confirmed that the Si—H bond had disappeared by means of an IR spectrum, and the reaction had proceeded. The reaction mixture was heated under reduced pressure to remove low-boiling components by distillation. Thereby, “Sugar Alcohol-Modified Silicone 1” represented by the following average compositional formula: MD₇₀D^{R*}₃M, wherein R*=xylitol residue, was obtained. The yield was 215.0 g (85%). The product had a kinetic viscosity of 83,000 mm²/sec or more at 25° C., and a refraction index of 1.415, and was in the form of a pale yellow opaque uniform viscous liquid.

### Reference Example 2

### Synthesis of Sugar Alcohol-Modified Silicone 2

93.1 g of a methylhydrogenpolysiloxane represented by the following average compositional formula: MD₃₆M^{H}, 8.6 g of xylitol monoallyl ether, and 30.0 g of isopropyl alcohol (IPA) were placed in a reactor, and the mixture was heated to 80° C. under a nitrogen stream while it was stirred. 12 mg of a platinum catalyst was added thereto, and the mixture was reacted for 2.5 hours at 80° C. It was confirmed that the Si—H bond had disappeared by means of an IR spectrum, and the reaction had proceeded. The reaction mixture was heated under reduced pressure to remove low-boiling components by distillation. Thereby, “Sugar Alcohol-Modified Silicone 2” represented by the following average compositional formula: MD₃₆M^{R*}, wherein R*=xylitol residue, was obtained. The yield was 89.4 g (88%). The product had a kinetic viscosity of 3,800 mm²/sec at 25° C., and a refraction index of 1.409, and was in the form of a pale yellow opaque uniform viscous liquid.

### Reference Example 3

### Synthesis of Tetraglycerol-Modified Silicone

168.9 g of a methylhydrogenpolysiloxane represented by the following average compositional formula: MD₇₂D^{H}₃M, 41.1 g of tetraglycerol monoallyl ether, 63.0 g of IPA were placed in a reactor, and the mixture was heated to 80° C. under a nitrogen stream while it was stirred. 24 mg of a platinum catalyst was added thereto, and the mixture was reacted for 3 hours at 80° C. It was confirmed that the Si—H bond had disappeared by means of an IR spectrum, and the reaction had proceeded. The reaction mixture was heated under reduced pressure to remove low-boiling components by distillation. Thereby, “Tetraglycerol-Modified Silicone” represented by the following average compositional formula: MD₇₂D^{R*2}₃M wherein R^{*2}=—C₃H₆O—X, wherein X represents a tetraglycerol moiety, was obtained. The yield was 185.2 g (88%). The product had a kinetic viscosity of 750,000 mm²/sec or more at 25° C., and a refraction index of 1.429, and was in the form of a milky opaque uniform viscous liquid.

### Example 1

### Preparation and Evaluation of “Powder Dispersion D1”

20 g of titanium oxide fine particles (trade name: MTY-100SAS, manufactured by Tayca Corporation, particle size=15 nm), 5 g of “Sugar Alcohol-Modified Silicone 1” obtained in Reference Example 1, and 25 g of decamethylcyclopentasiloxane (trade name: DC245, manufactured by Dow Corning Toray Co., Ltd.) were mixed, and 200 g of zirconia beads (diameter of 0.8 mm) were added thereto. The mixture was mixed for one hour by means of a paint shaker, and thereby, “Powder Dispersion D1” in the form of a slurry was prepared.

Next, the dispersing property (shearing rate dependency) and the dispersing property (stability over time) of the obtained “Powder Dispersion D1” were evaluated by the methods described below. The results are shown in Table 1. The aforementioned “Powder Dispersion D1” exhibited a low shearing rate dependency, and superior fluidity. In addition, the dispersion possessed superior properties in which thickening over time was reduced, and clumping of powders was reduced.

Evaluation of Dispersing Property (Shearing Rate Dependency)

A shearing rate dependency of a viscosity of a powder dispersion at 25° C. was determined in accordance with the equation described below. Change in viscosity in accordance with a shearing rate means that fluidity changes in accordance with clumping powders or the like. Low shearing rate dependency is preferred. A viscosity of a dispersion under a constant shearing rate was measured by means of an apparatus described below under measurement conditions.
Evaluation apparatus: Cone-plate viscometer AR1000-N, manufactured by TA Instruments Co., Ltd.
Measurement conditions: 40 mm, 1°, geometry made of steel

Dispersing property of powder dispersion (shearing rate dependency)=(Viscosity at a shearing rate of 10 sec⁻¹ (25° C.))/(Viscosity at a shearing rate of 100 sec⁻¹ (25° C.))

Evaluation of Dispersing Property (Stability Over Time)

The powder dispersion was allowed to stand for 2 weeks and for one month at 50° C., followed by returning the dispersion to 25° C. Subsequently, the viscosity of the powder dispersion was obtained in each case. The viscosity was obtained as a rotating viscosity at 25° C. measured by means of a Brookfield viscometer of the cone-plate type.

### Example 2

### Preparation and Evaluation of “Powder Dispersion D2”

20 g of titanium oxide fine particles (trade name: MTY-100SAS, manufactured by Tayca Corporation, particle size=15 nm), 5 g of “Sugar Alcohol-Modified Silicone 2” obtained in Reference Example 2, and 25 g of decamethylcyclopentasiloxane (trade name: DC245, manufactured by Dow Corning Toray Co., Ltd.) were mixed, and 200 g of zirconia beads (diameter of 0.8 mm) were added thereto. The mixture was mixed for one hour by means of a paint shaker, and thereby, “Powder Dispersion D2” in the form of a slurry was prepared.

Next, the dispersing property (shearing rate dependency) and the dispersing property (stability over time) of the obtained “Powder Dispersion D2” were evaluated by the methods described above. The results are shown in Table 1. The aforementioned “Powder Dispersion D2” exhibited an extremely low shearing rate dependency, and superior fluidity. In addition, the dispersion possessed superior properties in which thickening over time was reduced, and clumping of powders was reduced.

### Comparative Example 1

### Preparation and Evaluation of “Powder Dispersion D3”

20 g of titanium oxide fine particles (trade name: MTY-100SAS, manufactured by Tayca Corporation, particle size=15 nm), 5 g of a polyether-modified silicone (trade name: SS2910, manufactured by Dow Corning Toray Co., Ltd.), and 25 g of decamethylcyclopentasiloxane (trade name: DC245, manufactured by Dow Corning Toray Co., Ltd.) were mixed, and 200 g of zirconia beads (diameter of 0.8 mm) were added thereto. The mixture was mixed for one hour by means of a paint shaker, and thereby, “Powder Dispersion D3” in the form of a slurry was prepared.

Next, the dispersing property (shearing rate dependency) and the dispersing property (stability over time) of the obtained “Powder Dispersion D3” were evaluated by the methods described above. The results are shown in Table 1. The aforementioned “Powder Dispersion D3” exhibited a low shearing rate dependency, and superior fluidity was exhibited at the initial stage of the preparation, but thickening over time increased greatly. Clumping of powders over time occurred, and the fluidity thereof lost.

### Comparative Example 2

### Preparation and Evaluation of “Powder Dispersion D4”

20 g of titanium oxide fine particles (trade name: MTY-100SAS, manufactured by Tayca Corporation, particle size=15 nm), 5 g of “Tetraglycerol-Modified Silicone” obtained in Reference Example 3 and 25 g of decamethylcyclopentasiloxane (trade name: DC245, manufactured by Dow Corning Toray Co., Ltd.) were mixed, and 200 g of zirconia beads (diameter of 0.8 mm) were added thereto. The mixture was mixed for one hour by means of a paint shaker, and thereby, “Powder Dispersion D4” in the form of a slurry was prepared.

Next, the dispersing property (shearing rate dependency) and the dispersing property (stability over time) of the obtained “Powder Dispersion D4” were evaluated by the methods described above. The results are shown in Table 1. The aforementioned “Powder Dispersion D4” exhibited an extremely high shearing rate dependency, and poor fluidity even at the initial stage of the preparation.

TABLE 1

| | Example | | Comparative Example | |
|---|---|---|---|---|
| | 1 | 2 | 1 | 2 |
| | Powder | Powder | Powder | Powder |
| | Dispersion | Dispersion | Dispersion | Dispersion |
| | D1 | D2 | D3 | D4 |
| Shearing rate dependency | 9.7 | 1.5 | 1.1 | 25.0 |
| of the viscosity of the | | | | |
| dispersion | | | | |
| Viscosity (25° C.) of the | 1,300 | 36 | 61 | — |
| dispersion immediately | | | | |
| after preparing | | | | |
| (mPa · sec) | | | | |
| Viscosity (25° C.) of the | 350 | 38 | 1,220 | — |
| dispersion after standing | | | | |
| for two weeks at 50° C. | | | | |
| (mPa · sec) | | | | |
| Viscosity (25° C.) of the | 250 | 36 | 1,670 | — |
| dispersion after standing | | | | |
| for one month at 50° C. | | | | |
| (mPa · sec) | | | | |

### Examples 3 and 4 and Comparative Examples 3 and 4

### Preparation and Evaluation of Sunscreen Agent (Shaking Type)

First, components 1 to 12 in accordance with the composition shown in Table 2 were stirred and mixed well, and subsequently, a mixture of components 13 to 16 was added thereto, followed by further stirring and mixing. Subsequently, a container was filled with the aforementioned mixture. Thereby, a sunscreen agent of the shaking type was prepared. In the table, as the components indicated by Note 1 to Note 8, the components shown below were used. In the table, the numerical values were indicated by parts by weight (mass).

Note 1: Zinc Oxide Dispersion 1: 25 g of zinc oxide fine particles (trade name: FINEX-30S-LPT manufactured by Sakai Chemical Industry Co., Ltd., particle size=35 nm, 5 g of “Sugar Alcohol-Modified Silicone 1” obtained in Reference Example 1, and 20 g of decamethylcyclopentasiloxane (trade name: DC245, manufactured by Dow Corning Toray Co., Ltd.) were mixed, and 200 g of zirconia beads (diameter of 0.8 mm) were added thereto. The mixture was mixed for one hour by means of a paint shaker, and thereby, “Zinc Oxide Dispersion 1” in the form of a slurry was prepared.

Note 2: “Zinc Oxide Dispersion 2” was prepared in the same manner as described in the aforementioned Note 1, with the exception of using “Sugar Alcohol-Modified Silicone 2” obtained in Reference Example 2 instead of “Sugar Alcohol-Modified Silicone 1” obtained in Reference Example 1 in the aforementioned Note 1.

Note 3: “Zinc Oxide Dispersion 3” was prepared in the same manner as described in the aforementioned Note 1, with the exception of using “SS-2910”, manufactured by Dow Corning Toray Co., Ltd., instead of “Sugar Alcohol-Modified Silicone 1” obtained in Reference Example 1 in the aforementioned Note 1.

Note 4: “Zinc Oxide Dispersion 4” was prepared in the same manner as described in the aforementioned Note 1, with the exception of using “Tetraglycerol-Modified Silicone” obtained in Reference Example 3 instead of “Sugar Alcohol-Modified Silicone 1” obtained in Reference Example 1 in the aforementioned Note 1.

Note 5: Polyether-modified silicone (trade name: SS-2910, manufactured by Dow Corning Toray Co., Ltd.)

Note 6: Decamethylcyclopentasiloxane (trade name: DC245, manufactured by Dow Corning Toray Co., Ltd.)

Note 7: Dimethicone cross polymer (trade name: DC 9040 Silicone Elastomer Blend, manufactured by Dow Corning Corporation)

Note 8: Preservative (methylparaben)

Next, each category of “dispersion stability of powders in the preparation”, “stickiness of coating film” and “adhesive properties of coating film” of the obtained sunscreen agents was evaluated in accordance with the following evaluation methods and evaluation criteria. The results are shown in Table 2.

Dispersion Stability of Powders in the Preparation

After the sunscreen agent was prepared, the sunscreen agent was stored for one week at 50° C., and the viscosity increase of the sunscreen agent was evaluated.

Stickiness of Coating Film and Adhesive Properties of Coating Film

Ten panelists evaluated sensation during use of the coating films to be evaluated for the corresponding evaluation categories. Each of the panelists answered questionnaires so that with respect to each of the evaluation categories, in the case of evaluating as superior, 5 points were scored; in the case of evaluating as inferior, 1 point was scored; and in the case of evaluating as intermediate, 2 points, 3 points or 4 points were scored, and subsequently, the average thereof was used as an evaluation result of sensation during use. More particularly, with respect to stickiness of the coating film, the sunscreen agent was applied on a glass plate, followed by drying, and the feeling on touch thereof was evaluated by touching the applied agent. On the other hand, with respect to the adhesive properties of the coating film, the sunscreen agent was applied on a glass plate, followed by drying, and easiness of separation of the coating film was evaluated by rubbing the applied agent, in the same manner as described above. The results are shown in Table 2.

Each of the evaluation criteria in Table 2 was described below.

### Dispersion Stability

◯: Change of the viscosity of the sunscreen agent ≦±5%
Δ: ±5%<change of the viscosity of the sunscreen agent ≦±20%
X: ±20%<change of the viscosity of the sunscreen agent

### Stickiness

◯: Stickiness was not felt at all.
Δ: Stickiness was felt, but the level of the stickiness was hardly detectable.
X: Stickiness was clearly felt.

### Adhesive Properties

◯: Powders did not adhere to fingers.
Δ: Powders slightly adhered to fingers.
X: Powders clearly adhered to fingers.

TABLE 2

| | | | | Comparative | |
|---|---|---|---|---|---|
| | | Example | | Example | |
| | | 3 | 4 | 3 | 4 |
| 1 | Octyl methoxycinnamate | 10 | 10 | 10 | 10 |
| 2 | Titanium oxide dispersion | 0.4 | — | — | — |
| | (Powder Dispersion D1) | | | | |
| 3 | Titanium oxide dispersion | — | 0.4 | — | — |
| | (Powder Dispersion D2) | | | | |
| 4 | Titanium oxide dispersion | — | — | 0.4 | — |
| | (Powder Dispersion D3) | | | | |
| 5 | Titanium oxide dispersion | — | — | — | 0.4 |
| | (Powder Dispersion D4) | | | | |
| 6 | Zinc Oxide Dispersion 1 | 10 | — | — | — |
| | (Note 1) | | | | |
| 7 | Zinc Oxide Dispersion 2 | — | 10 | — | — |
| | (Note 2) | | | | |
| 8 | Zinc Oxide Dispersion 3 | — | — | 10 | — |
| | (Note 3) | | | | |
| 9 | Zinc Oxide Dispersion 4 | — | — | — | 10 |
| | (Note 4) | | | | |
| 10 | Polyether-modified silicone | 1.5 | 1.5 | 1.5 | 1.5 |
| | (Note 5) | | | | |
| 11 | Decamethylpentacyclo- | 18 | 18 | 18 | 18 |
| | siloxane (Note 6) | | | | |
| 12 | Dimethicone cross polymer | 3 | 3 | 3 | 3 |
| | (Note 7) | | | | |
| 13 | Preservative (Note 8) | 0.1 | 0.1 | 0.1 | 0.1 |
| 14 | Ethanol | 5 | 5 | 5 | 5 |
| 15 | 1,3-butylene glycol | 3 | 3 | 3 | 3 |
| 16 | Purified water | balance | balance | balance | balance |

| | | | | |
|---|---|---|---|---|
| Dispersion stability of powders | ◯ | ◯ | X | Δ |
| in the preparation (after one week | | | | |
| at 50° C.) | | | | |
| Stickiness of the coating film | ◯ | ◯ | X | Δ |
| Adhesive properties of the | ◯ | ◯ | Δ | Δ |
| coating film | | | | |

Among the aforementioned dispersions of titanium oxide, the aforementioned Powder Dispersion D1 and Powder Dispersion D2 were obtained by respectively using Sugar Alcohol-Modified Silicone 1 and Sugar Alcohol-Modified Silicone 2. Therefore, they correspond to the examples of the present invention. In the same manner as described above, among the aforementioned dispersions of zinc oxide, the dispersions indicated as Note 1 and Note 2 were also obtained by respectively using Sugar Alcohol-Modified Silicone 1 and Sugar Alcohol-Modified Silicone 2. Therefore, they also correspond to the examples of the present invention.

Example 3 and Comparative Example 3, as well as Example 4 and Comparative Example 4 were different only in view of the treatments of titanium oxide and zinc oxide with Sugar Alcohol-Modified Silicone 1 or the polyether-modified silicone, as well as the treatments with Sugar Alcohol-Modified Silicone 2 or the tetraglycerol-modified silicone, respectively. For this reason, the differences in the effects of the respective two results reflected the effects of the present invention.

The sunscreen agent containing the powder dispersion with the powder treatment agent of the present invention exhibited superior dispersion stability of powders and the coating film obtained by applying the sunscreen agent provided superior adhering properties without stickiness.

Hereinafter, the cosmetics of the present invention are described in detail with reference to Examples with detailed formulations. It should be understood that the cosmetics of the present invention are not restricted to the types and compositions described in the formulations shown in the Examples. The “part(s)” in the formulation means parts by weight (mass).

### Example 5

### W/O Type Foundation (Gel)

| | |
|---|---|
| (Components) | (part(s)) |

| | | |
|---|---|---|
| 1. | Titanium oxide with octylsilane | 5 |
| 2. | Iron oxide (yellow, red, and black) | 0.5 |
| | treated with octylsilane | |
| 3. | Spherical polystyrene (particle size = 3 μm) | 3 |
| 4. | Sorbitan sesquioleate | 1 |
| 5. | Cyclopentasiloxane/(acrylate/ | 20 |
| | polytrimethylsiloxy methacrylate) copolymer | |
| | (Note 1) | |
| 6. | Trimethylsiloxysilicic acid | 4 |
| 7. | Sugar Alcohol-Modified Silicone 1 | 5 |
| 8. | Isodecyl neopentanoate | 5 |
| 9. | Isopropyl palmitate | 2 |
| 10. | Vitamin E acetate | 0.1 |
| 11. | Perfume | q.s. |
| 12. | Ethanol | 10 |
| 13. | Dipropylene glycol | 5 |
| 14. | Preservative | q.s. |
| 15. | 1,3-propanediol | 3 |
| 16. | Purified water | remainder |

(Note 1): FA4001CM Silicone Acrylate, manufactured by Dow Corning Toray Co., Ltd., was used.

Preparation Method

Step 1: Components 1 to 3 were stirred and mixed in a mixer.
Step 2: Components 4 to 11 were stirred and mixed. The mixture obtained in the aforementioned step 1 was added thereto under stirring, followed by further stirring and mixing.
Step 3: Components 12 to 16 were uniformly dissolved, and the mixture obtained by the aforementioned step 2 was added thereto under stirring, followed by further stirring and mixing. A container was charged with the mixture. Thereby, a product was obtained.

The foundation of Example 5 possessed a refreshing feeling on touch. In addition, a texture was not noticeable, good spreading properties were exhibited, and superior stability of the formulation was provided.

### Example 6

### W/O Type Foundation (Liquid)

| (Components) | | (part(s)) |
|---|---|---|
| 1. | Decamethylcyclopentasiloxane | 10 |
| 2. | Dimethylpolysiloxane (2 mm²/s) | 5 |
| 3. | Dimethylpolysiloxane (6 mm²/s) | 8 |
| 4. | Pentaerythritol rosinate | 2 |
| 5. | Di-2-ethylhexyl succinate | 7 |
| 6. | Octyl paramethoxycinnamate | 1 |
| 7. | Polyether-modified silicone (Note 1) | 3 |
| 8. | Decamethylcyclopentasiloxane-dimethicone | 2 |
| | cross polymer (Note 2) | |
| 9. | Sugar Alcohol-Modified Silicone 1 | 2 |
| 10. | Fine particulate titanium oxide treated | 5 |
| | with silicone | |
| 11. | Titanium oxide treated with silicone | 7 |
| 12. | Spherical silica treated with silicone | 2 |
| 13. | Red iron oxide treated with silicone | 0.5 |
| 14. | Yellow iron oxide treated with silicone | 1.4 |
| 15. | Black oxide treated with silicone | 0.1 |
| 16. | 1,3-butylene glycol | 3 |
| 17. | Ethanol | 7 |
| 18. | Preservative | q.s |
| 19. | Purified water | remainder |
| 20. | Perfume | q.s. |

(Note 1): SS-2910, manufactured by Dow Corning Toray Co., Ltd., was used.
(Note 2): DC9040, manufactured by Dow Corning Corporation, was used.

Preparation Method

Step 1: Components 1 to 9 were stirred and mixed.
Step 2: Components 10 to 15 were stirred and mixed by means of a mixer.
Step 3: The mixture obtained in the aforementioned step 2 was added to the mixture obtained in the aforementioned step 1, followed by further stirring and mixing.
Step 4: An aqueous phase obtained by uniformly dissolving components 16 to 20 was added to the mixture obtained in the aforementioned step 3 to emulsify them. A container was charged with the emulsion, and thereby, a product was obtained.

The W/O type liquid foundation exhibited superior emulsification stability during use, provided superior water resistance and superior makeup durability, and exhibited superior spreading properties and adhesiveness without noticeable texture and wrinkles.

### Example 7

### Mascara

| (Components) | | (part(s)) |
|---|---|---|
| 1. | Paraffin wax | 5 |
| 2. | Light liquid isoparaffin | remainder |
| 3. | Caprylmethicone | 0.5 |
| 4. | Sugar Alcohol-Modified Silicone 1 | 0.5 |
| 5. | Trioctanoin | 13 |
| 6. | Decamethylcyclopentasiloxane | 20 |
| 7. | Inulin stearate | 5 |
| 8. | Cyclopentasiloxane-dimethicone cross | 10 |
| | polymer (Note 1) | |
| 9. | Black iron oxide surface-treated with | 6 |
| | a fluorine compound | |
| 10. | Ester of a sugar fatty acid | 4 |
| 11. | Beeswax | 5 |
| 12. | Pentaerythritol rosinate | 5 |
| 13. | Preservative | q.s. |
| 14. | Purified water | 5 |

(Note 1): DC9040, manufactured by Dow Corning Corporation was used.

Preparation Method

Components 1 to 12 were heated and dissolved. Subsequently, the solution was sufficiently mixed and dispersed. A mixture of components 13 and 14 was added thereto to emulsify them. A container was charged with the emulsion. Thereby, a product was obtained.

The mascara of Example 7 possessed a dark black outer appearance during use, and exhibited superior glossiness. In addition, good adhering properties with respect to eyelashes and superior effects of curling and increasing the volume of eyelashes were exhibited. Superior durability thereof was provided.

### Example 8

### Foundation Base Cream

| (Components) | | (part(s)) |
|---|---|---|
| 1. | Dimethylpolysiloxane (2 mm²/s) | 2 |
| 2. | Decamethylcyclopentasiloxane | 10 |
| 3. | Polyether-modified silicone (Note 1) | 3 |
| 4. | Cetyl isooctanoate | 5 |
| 5. | Sugar Alcohol-Modified Silicone 2 | 3 |
| 6. | 2-ethylhexyl paramethoxycinnamate | 2 |
| 7. | Silicone elastomer (Note 2) | 4 |
| 8. | Organo-modified bentonite | 0.5 |
| 9. | Barium sulfate | 2 |
| 10. | Talc | 1 |
| 11. | Nylon powder | 3 |
| 12. | Preservative | q.s. |
| 13. | Xanthan gum | 0.1 |
| 14. | Magnesium L-ascorbyl phosphate | 0.3 |
| 15. | Purified water | q.s. |

(Note 1): SS-2910, manufactured by Dow Corning Toray Co., Ltd., was used.
(Note 2): 9045 Silicone Elastomer Blend, manufactured by Dow Corning Corporation, was used.

Preparation Method

Components 1 to 11 were mixed and dispersed, and subsequently, a mixture of components 12 to 15 was added thereto to emulsify them at room temperature. A container was charged with the emulsion, and thereby, a product was obtained.

The foundation base cream of Example 8 exhibited good spreading properties and provided superior uniformity of the cosmetic film and superior adhesiveness to the skin. In addition, the noticeable skin texture, wrinkles and pores were reduced. In addition, a stable emulsion state was achieved.

### Example 9

### Lip Gloss

| (Components) | | (Part(s)) |
|---|---|---|
| 1. | Polyamide-modified silicone (Note 1) | 10 |
| 2. | Sugar Alcohol-Modified Silicone 1 | 5 |
| 3. | Methylphenyl-modified silicone | 28 |
| 4. | Isodecyl isononanoate | 38 |
| 5. | Isohexadecane | 14 |
| 5. | Trioctanoin | 2 |
| 6. | Titanium mica | 3 |

(Note 1): 2-8178 Gellant, manufactured by Dow Corning Corporation, was used.

Preparation Method

All components were heated and mixed at 100° C., and subsequently, a container was charged with the mixture. Thereby, a product was obtained.

The lip gloss of Example 9 exhibited good compatibility with the oil-based raw materials, and good storage stability in the case of storing the product was provided.

### Example 10

### Lipstick

| (Components) | | (part(s)) |
|---|---|---|
| 1. | Triethylhexanoin | 10.0 |
| 2. | Cetyl hexanoate | 17.0 |
| 3. | Sorbitan sesquiisostearate | 4.0 |
| 4. | Microcrystalline wax | 10.0 |
| 5. | Paraffin wax | 15.0 |
| 6. | Diisostearyl malate | 7.0 |
| 7. | Glyceryl triisostearate | 9.0 |
| 8. | Propylene glycol dicaprate | 7.0 |
| 9. | Inulin stearate (product name: | 2.0 |
| | Rheopearl ISL2, manufactured by Chiba | |
| | Flour Milling Co., Ltd.) | |
| 10. | Sugar Alcohol-Modified Silicone 1 | 3.0 |
| 11. | Isododecane/(acrylate/polytrimethylsiloxy | 3.0 |
| | methacrylate) copolymer(Note 1) | |
| 12. | DC 593(Note 2) | 2.0 |
| 13. | Yellow No. 4 | q.s. |
| 14. | Titanium oxide | 1.0 |
| 15. | Black iron oxide | 1.0 |
| 16. | Mica | 1.0 |
| 17. | Red No. 104 | q.s. |
| 18. | Purified water | 7.0 |
| 19. | 1,3-butylene glycol | 1.0 |
| 20. | Preservative | q.s. |
| 21. | Perfume | q.s. |

(Note 1) Isododecane solution of (acrylate/polytrimethylsiloxy methacrylate) copolymer (active component = 40% by weight)
(Note 2) Dimethylpolysiloxane (100 cst) solution of trimethylsiloxysilicic acid (active component = 33%)

Preparation Method

Components 1 to 17 were heated and dissolved. Separately, Components 18 to 20 were uniformly mixed, and the obtained mixture was added to the mixture of components 1 to 17. The whole mixture was further stirred and mixed. Finally, component 22 was added thereto. An airtight container was charged with the resultant mixture. Thereby, a product was obtained.

The lipstick of Example 10 provided a high grade sensation and good spreading properties, could be uniformly applied to the lips, and provided superior makeup with superior glossiness and a superior transparent impression. In addition, after the lipstick was applied to lips, stickiness on the lips was not exhibited and good storage stability in the case of storing the product was provided.

### Example 11

### Sunscreen Cream (W/O Type)

| (Components) | | (part(s)) |
|---|---|---|
| 1. | Octyl methoxycinnamate | 7.5 |
| 2. | Cyclopentasiloxane | 8 |
| 3. | Dimethicone (6 cs) | 2 |
| 4. | Cyclopentasiloxane/(acrylate/ | 1 |
| | polytrimethylsiloxy methacrylate) copolymer | |
| | (Note 1) | |
| 5. | Dimethicone cross polymer (Note 2) | 2 |
| 6. | Polysilicone 13 (Note 3) | 1 |
| 7. | Sugar Alcohol-Modified Silicone 2 | 1 |
| 8. | Dispersion of titanium oxide (40%) (Note 4) | 2 |
| 9. | Dispersion of zinc oxide (55%) (Note 5) | 10 |
| 10. | Sodium chloride | 1 |
| 11. | Panthenol | 0.5 |
| 12. | Purified water | remainder |
| 13. | Glycerol | 2 |
| 14. | Preservative | 0.05 |

(Note 1): FA40001CM Silicone Acrylate, manufactured by Dow Corning Toray Co., Ltd., was used.
(Note 2): DC9041, manufactured by Dow Corning Toray Co., Ltd., was used.
(Note 3): A polyether-modified silicon of a linear block copolyment (ABn) type (FZ-2233, manufactured by Dow Corning Toray Co., Ltd.) was used.
(Note 4): A dispersion was prepared using the same composition as described in “Powder disperision D1” of Example 1, with the exception of using “Sugar alcohol-modified silicon 2” as a dispersing agent.
(Note 5): A dispersion was prepared by using a composition of 50 g of fine particles of zinc oxide, 5 g of “Sugar Alcohol-Modified Silicone 2”, and 45 g of decamethylpentacyclosiloxane, in accordance with the same method as described in Example 1, with the exception of using “Sugar Alcohol-Modified Silicone 2” as a dispersing agent.

Preparation Method

Step 1: Components 1 to 9 were mixed.

Step 2: Components 10 to 14 were mixed.

Step 3: The aqueous phase obtained in the aforementioned step 2 was added to the mixture obtained in the aforementioned step 1 under stirring to emulsify them. Subsequently, a container was charged therewith, and thereby, a product was obtained.

Example 11 relates to a sunscreen cream containing a dispersion of an inorganic UV controlling component treated with Sugar Alcohol-Modified Silicone 2 according to the present invention. While the sunscreen cream of Example 11 contained a large amount of the aqueous phase components and the inorganic UV controlling component, the sunscreen cream could be stored for a long time at about 40° C. (temperature during the summer season) without any separation of the oil-based components or powders, and superior stability over time was exhibited. In addition, a good spreading property and a superior sensation during use with reduced stickiness were exhibited at the time of use. A durable UV-protection effect was provided without irritation. No change of the aforementioned good sensation during use was observed before or even after storing at about 40° C.

1-15. (canceled)

16. A method comprising:
providing a powder treatment agent comprising a sugar alcohol-modified organopolysiloxane wherein a sugar alcohol-containing organic group represented by general formula (1-1): 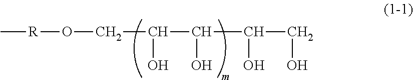
wherein
R represents a divalent organic group; and
m is 1 or 2,
or represented by general formula (1-2): 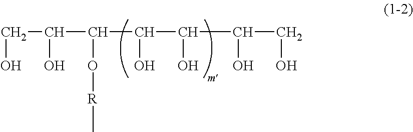
wherein
R is the same as defined above; and
m′ is 0 or 1,
is bonded to a silicon atom of the sugar alcohol-modified organopolysiloxane; and treating a powder with the powder treatment agent.

17. The method according to claim 16, wherein in said general formula (1-1) or (1-2), R is a substituted or non-substituted, and linear or branched divalent hydrocarbon group having 3 to 5 carbon atoms.

18. The method according to claim 16, wherein said sugar alcohol-modified organopolysiloxane is represented by average formula:
XₓR¹_{y}SiO_{(4-x-y)/2}
wherein
each X independently represents a sugar alcohol-containing organic group represented by said general formula (1-1) or (1-2);
each R¹ independently represents a monovalent organic group, with the proviso that the one represented by X is excluded therefrom; and
x and y are numbers independently satisfying 0<x<1, 0<y<3, and 0<x+y<4.

19. The method according to claim 18, wherein R¹ in said average formula represents a substituted or non-substituted, and linear or branched monovalent hydrocarbon group having 1 to 8 carbon atoms, a polyoxyalkylene group represented by the following formula: —R²O(AO)ₙR³ wherein n=1 to 100; AO represents an oxyalkylene group having 2 to 4 carbon atoms; R² represents a substituted or non-substituted, and linear or branched divalent hydrocarbon group having 3 to 5 carbon atoms; R³ represents a hydrogen atom, a substituted or non-substituted, and linear or branched monovalent hydrocarbon group having 1 to 8 carbon atoms, or a substituted or non-substituted, and linear or branched acyl group having 2 to 8 carbon atoms; an alkoxy group having 1 to 8 carbon atoms, a hydroxyl group or a hydrogen atom, with the proviso that all R¹s do not represent a hydroxyl group, a hydrogen atom, said alkoxy group or said polyoxyalkylene group.

20. The method according to claim 18, wherein said sugar alcohol-modified organopolysiloxane is represented by general formula (2): 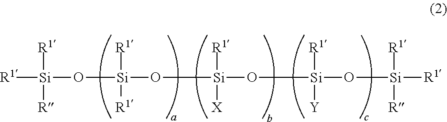
wherein
each X independently represents a sugar alcohol-containing organic group represented by said formula (1-1) or (1-2);
each R¹ independently represents a substituted or non-substituted, and linear or branched monovalent hydrocarbon group having 1 to 8 carbon atoms;
each Y independently represents a polyoxyalkylene group represented by the following formula: —R⁴O(AO)ₙR⁵ wherein AO represents an oxyalkylene group having 2 to 4 carbon atoms; R⁴ represents a substituted or non-substituted, and linear or branched divalent hydrocarbon group having 3 to 5 carbon atoms; R⁵ represents a hydrogen atom, a substituted or non-substituted, and linear or branched monovalent hydrocarbon group having 1 to 8 carbon atoms, or a substituted or non-substituted, and linear or branched acyl group having 2 to 8 carbon atoms; and n=1 to 100;
each R″ independently represents any one of the functional groups represented by said R¹, X or Y;
a=0 to 700; b=0 to 100; and c=0 to 50, with the proviso that in the case of b=0, at least one R″ is X.

21. The method according to claim 20, wherein said sugar alcohol-modified organopolysiloxane is represented by general formula (2′): 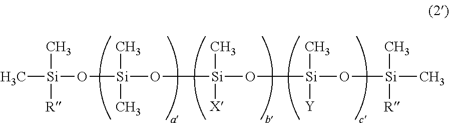
wherein
each X′ independently represents a sugar alcohol-containing organic group represented by general formula (1′-1): 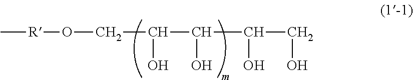
wherein
R′ represents a substituted or non-substituted, and linear or branched alkylene group having 3 to 5 carbon atoms; and m is 1 or 2,
or represented by general formula (1′-2): 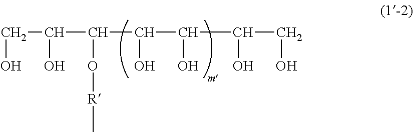
wherein
R′ is the same as defined above; and m′ is 0 or 1;
each Y independently represents a polyoxyalkylene group represented by formula: —R⁴O(AO)ₙR⁵ wherein AO represents an oxyalkylene group having 2 to 4 carbon atoms; R⁴ represents a substituted or non-substituted, and linear or branched divalent hydrocarbon group having 3 to 5 carbon atoms; R⁵ represents a hydrogen atom, a substituted or non-substituted, and linear or branched monovalent hydrocarbon group having 1 to 8 carbon atoms, or a substituted or non-substituted, and linear or branched acyl group having 2 to 8 carbon atoms; and n=1 to 100;
each R″ independently represents any one of a methyl group, a group represented by any of X′ and a group represented by any of Y;
a′=0 to 250; b′=0 to 50; c′=0 to 25; and a′+b′+c′=0 to 250, with the proviso that in the case of b′=0, at least one R″ is X′.

22. The method according to claim 16, wherein said powder treatment agent is a powder-surface treatment agent.

23. The method according to claim 22, wherein the powder comprises a surface and wherein treating the powder comprises treating a surface of the powder with the powder-surface treatment agent.

24. The method according to claim 23, wherein the powder is comprised in a cosmetic raw material.

25. The method according to claim 24, wherein the cosmetic raw material further comprises at least one oil agent, wherein the oil agent is in the form of a liquid at 5 to 100° C., and is selected from the group consisting of a silicone oil, a non-polar organic compound and a low-polar organic compound.

26. The method according to claim 23, wherein the powder is comprised in a cosmetic.

27. The method according to claim 24, wherein the cosmetic raw material is comprised in a cosmetic.

28. The method according to claim 26, wherein the cosmetic further comprises at least one oil agent, wherein the oil agent is in the form of a liquid at 5 to 100° C., and is selected from the group consisting of a silicone oil, a non-polar organic compound and a low-polar organic compound.

29. The method according to claim 28, wherein the cosmetic further comprises water.

30. The method according to claim 29, wherein the cosmetic is in the form of an oil-in-water emulsion or a water-in-oil emulsion.
